(19) United States
(12) Patent Application Publication
(10) Pub. No.: US 2010/0068152 A1
(43) Pub. Date: Mar. 18, 2010

(54) Ex vivo modifiable particle or polymeric based final dosage form

(75) Inventors: Mahalaxmi Gita Bangera, Renton, WA (US); Edward S. Boyden, Cambridge, MA (US); Roderick A. Hyde, Redmond, WA (US); Muriel Y. Ishikawa, Livermore, CA (US); Dennis J. Rivet, Portsmouth, VA (US); Elizabeth A. Sweeney, Seattle, WA (US); Lowell L. Wood, JR., Bellevue, WA (US); Victoria Y.H. Wood, Livermore, CA (US)

Correspondence Address: SEARETE LLC;CLARENCE T. TEGREENE, 1756 - 114TH AVE., S.E., SUITE 110, BELLEVUE, WA, 98004, US

(73) Assignee: Searete LLC, a limited liability corporation of the State of Delaware

(21) Appl. No.: 12/322,877

(22) Filed: Feb. 5, 2009

Related U.S. Application Data
(63) Continuation-in-part of application No. 12/284,015, filed on Sep. 16, 2008.
Application No. 12/284,015 is a continuation-in-part of application No. 12/284,014, filed on Sep. 16, 2008.
Application No. 12/284,014 is a continuation-in-part of application No. 12/284,013, filed on Sep. 16, 2008.

(51) Int. Cl. A61K 9/14; A61K 9/10; A61K 9/127

(52) U.S. Cl. 424/10.1; 424/490; 424/484; 424/450

(57) ABSTRACT

Provided embodiments include a final dosage form, an article of manufacture, and method. A final dosage form for administering a medicament to an animal is provided. The final dosage form includes the medicament, and a particle or polymeric material. The particle or polymeric material carries the medicament and is configured in a medicament-retention state. In medicament-retention state, the medicament is substantially not bioavailable to the animal if the final dosage form is administered to the animal. The particle or polymeric material is modifiable ex vivo by an exposure to a stimulus to a medicament-release state. In the medicament-release state, the medicament is substantially bioavailable to the animal if the final dosage form is administered to the animal.

### CROSS-REFERENCE TO RELATED APPLICATIONS

The present application is related to and claims the benefit of the earliest available effective filing date(s) from the following listed application(s) (the “Related Applications”) (e.g., claims earliest available priority dates for other than provisional patent applications or claims benefits under 35 USC § 119(e) for provisional patent applications, for any and all parent, grandparent, great-grandparent, etc. applications of the Related Application(s)).

### RELATED APPLICATIONS

For purposes of the USPTO extra-statutory requirements, the present application constitutes a continuation-in-part of U.S. patent application Ser. No. 12/284,015, titled INDIVIDUALIZABLE DOSAGE FORM, naming Mahalaxmi Gita Bangera, Edward S. Boyden, Roderick A. Hyde, Muriel Y. Ishikawa, Dennis J. Rivet, Elizabeth A. Sweeney, Lowell L. Wood, Jr., and Victoria Y. H. Wood as inventors, filed Sep. 16, 2008, which is currently co-pending, or is an application of which a currently co-pending application is entitled to the benefit of the filing date.

For purposes of the USPTO extra-statutory requirements, the present application constitutes a continuation-in-part of U.S. patent application Ser. No. 12/284,014, titled PERSONALIZABLE DOSAGE FORM, naming Mahalaxmi Gita Bangera, Edward S. Boyden, Roderick A. Hyde, Muriel Y. Ishikawa, Dennis J. Rivet, Elizabeth A. Sweeney, Lowell L. Wood, Jr., and Victoria Y. H. Wood as inventors, filed Sep. 16, 2008, which is currently co-pending, or is an application of which a currently co-pending application is entitled to the benefit of the filing date.

For purposes of the USPTO extra-statutory requirements, the present application constitutes a continuation-in-part of U.S. patent application Ser. No. 12/284,013, titled MODIFIABLE DOSAGE FORM, naming Mahalaxmi Gita Bangera, Edward S. Boyden, Roderick A. Hyde, Muriel Y. Ishikawa, Dennis J. Rivet, Elizabeth A. Sweeney, Lowell L. Wood, Jr., and Victoria Y. H. Wood as inventors, filed Sep. 16, 2008, which is currently co-pending, or is an application of which a currently co-pending application is entitled to the benefit of the filing date.

For purposes of the USPTO extra-statutory requirements, the present application constitutes a continuation-in-part of United States patent application No. To Be Assigned, titled EX VIVO ACTIVATABLE FINAL DOSAGE FORM, naming Mahalaxmi Gita Bangera, Edward S. Boyden, Roderick A. Hyde, Muriel Y. Ishikawa, Dennis J. Rivet, Elizabeth A. Sweeney, Lowell L. Wood, Jr., and Victoria Y. H. Wood as inventors, filed Feb. 5, 2009, which is currently co-pending, or is an application of which a currently co-pending application is entitled to the benefit of the filing date.

For purposes of the USPTO extra-statutory requirements, the present application constitutes a continuation-in-part of United States patent application No. To Be Assigned, titled MODIFYING A MEDICAMENT AVAILABLILITY STATE OF A FINAL DOSAGE FORM, naming Mahalaxmi Gita Bangera, Edward S. Boyden, Roderick A. Hyde, Muriel Y. Ishikawa, Dennis J. Rivet, Elizabeth A. Sweeney, Lowell L. Wood, Jr., and Victoria Y. H. Wood as inventors, filed Feb. 5, 2009, which is currently co-pending, or is an application of which a currently co-pending application is entitled to the benefit of the filing date.

The United States Patent Office (USPTO) has published a notice to the effect that the USPTO's computer programs require that patent applicants reference both a serial number and indicate whether an application is a continuation or continuation-in-part. Stephen G. Kunin, Benefit of Prior-Filed Application, USPTO Official Gazette Mar. 18, 2003, available at http://www.uspto.gov/web/offices/com/sol/og/2003/week11/patbene.htm. The present Applicant Entity (hereinafter “Applicant”) has provided above a specific reference to the application(s) from which priority is being claimed as recited by statute. Applicant understands that the statute is unambiguous in its specific reference language and does not require either a serial number or any characterization, such as “continuation” or “continuation-in-part,” for claiming priority to U.S. patent applications. Notwithstanding the foregoing, Applicant understands that the USPTO's computer programs have certain data entry requirements, and hence Applicant is designating the present application as a continuation-in-part of its parent applications as set forth above, but expressly points out that such designations are not to be construed in any way as any type of commentary or admission as to whether or not the present application contains any new matter in addition to the matter of its parent application(s).

All subject matter of the Related Applications and of any and all parent, grandparent, great-grandparent, etc. applications of the Related Applications is incorporated herein by reference to the extent such subject matter is not inconsistent herewith.

### SUMMARY

An embodiment of the subject matter described herein provides a final dosage form for administering a medicament to an animal. The final dosage form includes an outer layer, the medicament, and a release element. The release element configured in a medicament-holding state and modifiable ex vivo to a medicament-discharge state by an exposure to a stimulus. In an embodiment, the final dosage form includes a chamber substantially within the outer layer and carrying the medicament. In an embodiment, the final dosage form includes an indicator element configured to indicate an exposure of the release element to the stimulus.

Another embodiment of the subject matter described herein provides a final dosage form for administering a medicament to an animal. The final dosage form includes a release element configured in a medicament-holding state and modifiable ex vivo to a medicament-discharge state by an exposure to a stimulus. The final dosage form also includes a site carrying the medicament, the medicament, and a containment element. The containment element retains the medicament within the final dosage form until the final dosage form is administered to the animal. In an embodiment, the final dosage form further includes an indicator element configured to indicate an exposure of the release element to the stimulus. In an embodiment, the final dosage form further includes an outer layer surrounding the release element.

A further embodiment of the subject matter described herein provides an article of manufacture. The article includes at least one final dosage form for administering a therapeutically effective amount of a medicament to an animal, and instructions specifying the ex vivo exposure of a release element of the final dosage form to a stimulus sufficient to modify the release element to the medicament-discharge state. The final dosage form includes an outer layer, the release element, a site carrying the medicament, and the medicament. The release element is configured in a medicament-holding state and modifiable ex vivo to a medicament-discharge state by an exposure to a stimulus.

An embodiment provides a method of modifying a medicament availability characteristic of a final dosage form. The method includes initiating an ex vivo exposure of a release element of the final dosage form to a stimulus. The initiated stimulus is selected to transform the release element from a medicament-holding state to a medicament-discharge state. The final dosage form includes an outer layer, a site carrying the medicament, the medicament, and the release element. The release element is configured in the medicament-holding state wherein a medicament is substantially not bioavailable to the animal. The release element is modifiable ex vivo to the medicament-discharge state by the exposure to the stimulus wherein the medicament is substantially bioavailable to the animal. In an embodiment, the final dosage form further includes a containment element retaining the medicament within the final dosage form until the final dosage form is introduced into the animal. In an embodiment, the final dosage form further includes an indicator element configured to indicate an exposure of the release element to the stimulus. In an embodiment, the initiating an ex vivo exposure of a release element of the final dosage form to a stimulus includes initiating a first ex vivo exposure of a release element of the final dosage form to a stimulus, the first initiated stimulus selected to transform the release element from a medicament-holding state to a medicament-discharge state. This embodiment further includes receiving an indication of a first ex vivo exposure of the release element of the final dosage form to the first initiated stimulus. The indication is generated in response to an indicator element of the final dosage form configured to indicate an exposure of the release element to the stimulus. This embodiment further includes initiating a second ex vivo exposure of the release element of the final dosage form to the stimulus. The second initiated ex vivo exposure stimulus is selected to further transform the release element from the medicament-holding state to the medicament-discharge state.

Another embodiment provides a final dosage form for administering a medicament to an animal. The final dosage form includes means for protecting the final dosage form from an ex vivo environment. The final dosage form also includes means for releasing the medicament that is configured in a medicament-holding state, and that is modifiable ex vivo to a medicament-discharge state by an exposure to a stimulus. The final dosage form further includes the medicament. In an embodiment, the final dosage form further includes means for carrying the medicament. In an embodiment, the final dosage form further includes means for indicating an exposure of the means for releasing the medicament to the stimulus. In an embodiment, the final dosage form further includes means for containing the medicament within the final dosage form until the final dosage form is introduced into the animal.

A further embodiment provides a final dosage form for administering a medicament to an animal. The final dosage form includes the medicament, and a particle or polymeric material carrying the medicament. The particle or polymeric material is configured in a medicament-retention state wherein the medicament is substantially not bioavailable to the animal if the final dosage form is administered to the animal. The particle or polymeric material is modifiable ex vivo by an exposure to a stimulus to a medicament-release state wherein the medicament is substantially bioavailable to the animal if the final dosage form is administered to the animal. In an embodiment, the final dosage form further includes a transport medium suitable for delivering the particle or polymeric material carrying the medicament to the animal. In an embodiment, the final dosage form further includes an indicator substance configured to indicate an exposure of the particle or polymeric substance to the stimulus.

An embodiment provides an article of manufacture. The article of manufacture includes at least one final dosage form for administering a medicament to an animal, and an instruction. The final dosage form includes the medicament, and a particle or polymeric material. The particle or polymeric material carries the medicament in a medicament-retention state wherein the medicament is substantially not bioavailable to the animal after administration of the final dosage form. The particle or polymeric material is modifiable ex vivo to a medicament-release state by an exposure to a stimulus wherein the medicament is substantially bioavailable to the animal after administration of the final dosage form. The instruction includes an instruction for the ex vivo exposure of the particle or polymeric material to a human-initiated stimulus sufficient to transform the particle or polymeric material to allow a discharge of at least a portion of the therapeutically effective amount of the medicament from the particle or polymeric carrier. In an embodiment, the article of manufacture further includes label associated the at least one final dosage form and providing the instruction. In an embodiment, the article of manufacture further includes an insert into a package containing the at least one final dosage form and providing the instruction. In an embodiment, the article of manufacture further includes a transport medium suitable for administering the particle or polymeric material carrying the medicament to the animal. In an embodiment, the final dosage form further includes an indicator substance configured to indicate an exposure to the stimulus of the particle or polymeric material.

Another embodiment provides a final dosage for administering a medicament to an animal. The final dosage form includes at least one molecule of the medicament and a particle or polymeric carrier. The particle or polymeric carrier is operable to bind the at least one molecule of the medicament. The particle or polymeric carrier is configured in a first medicament-bioavailability state. The particle or polymeric carrier is modifiable ex vivo to a second medicament-bioavailability state by an exposure to a stimulus. In an embodiment, the final dosage form of further includes a transport medium suitable for delivering the particle or polymeric carrier holding the at least one molecule of the medicament to the animal. In an embodiment, the final dosage form further includes an indicator substance configured to visually indicate an exposure of the particle or polymeric carrier holding the at least one molecule of the medicament to the stimulus.

A further embodiment provides a method of modifying a medicament availability state of a final dosage form. The method includes initiating an ex vivo exposure of a particle or polymeric material of the final dosage form to a stimulus. The initiated stimulus is selected to transform the particle or polymeric material from a medicament-retention state to a medicament-release state. The final dosage form includes the medicament and the particle or polymeric material. The particle or polymeric material carries the medicament in the medicament-retention state. In the medicament-retention state, the medicament is substantially not bioavailable if the final dosage form is administered to the animal. The particle or polymeric material is transformable ex vivo to the medicament-release state by the exposure to a stimulus. In the medicament-release state the medicament is substantially bioavailable if the final dosage form is administered to the animal. In an embodiment, the final dosage form further includes a containment element retaining the medicament within the final dosage form until the final dosage form is introduced into the animal. In an embodiment, the final dosage form further includes an indicator element configured to indicate an exposure of the particle or polymeric material to the stimulus. In an embodiment, the ex vivo exposure of a particle or polymeric material of the final dosage form to a stimulus includes initiating a first ex vivo exposure of a particle or polymeric material of the final dosage form to a stimulus, the first initiated stimulus selected to transform the particle or polymeric material from a medicament-retention state to a medicament-release state. This embodiment further includes receiving an indication of the first ex vivo exposure of the release element of the final dosage form to the stimulus, the indication generated in response to an indicator element of the final dosage form configured to indicate an exposure of the release element to the stimulus. This embodiment also includes initiating a second ex vivo exposure of the release element of the final dosage form to the stimulus. The initiated second ex vivo exposure stimulus is selected to further transform the release element from the medicament-holding state to the medicament-discharge state.

An embodiment provides a final dosage form for administering a medicament to an animal. The final dosage form includes means for entrapping at least one molecule of the medicament. The final dosage form also includes means for controlling an availability of the entrapped at least one molecule of medicament, wherein the entrapped at least one molecule of medicament is initially substantially not bioavailable if the final dosage form is administered to the animal, and wherein the availability of the entrapped medicament is modifiable ex vivo by an exposure to a stimulus to be substantially bioavailable if the final dosage form is administered to the animal. The final dosage form further includes means for protecting the means for entrapping at least one molecule of the medicament from an ex vivo environment of the final dosage form. The final dosage form also includes the medicament. In an embodiment, the final dosage form further includes means for indicating an exposure to the stimulus by the means for controlling an availability of the entrapped at least one molecule of medicament. In an embodiment, the final dosage form further includes means for containing the medicament within the final dosage form before the final dosage form is administered to the animal. In an embodiment, the final dosage form further includes means for carrying the final dosage form into the animal.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 illustrates an example environment that includes an animal, a cross-sectional view of an example final dosage form for administering a medicament to the animal, and an example stimulation source operable to emit a stimulus;

FIG. 2 illustrates another example environment that includes the animal, a cross-sectional view of an example final dosage form for administering a medicament to the animal, and the example stimulation source operable to emit the stimulus;

FIG. 3 illustrates a further example environment that includes the animal, a cross-sectional view of an example final dosage form for administering a medicament to the animal, and the example stimulation source operable to emit the stimulus;

FIG. 4 illustrates another example environment that includes the animal, a cross-sectional view of an example final dosage form for administering a medicament to the animal, and the example stimulation source operable to emit the stimulus;

FIG. 5 illustrates a further example environment that includes an animal, a cross-sectional view of an example final dosage form for transporting medicament to the animal;

FIG. 6 illustrates an example environment that includes an article of manufacture;

FIG. 7 illustrates an example operational flow modulating a medicament-release characteristic of a final dosage form;

FIG. 8 illustrates an alternative embodiment of the operational flow of FIG. 7;

FIG. 9 illustrates an example operational flow fulfilling a request specifying a dose of a medicament for an individual animal;

FIG. 10 illustrates an alternative embodiment of the example operational flow of FIG. 9;

FIG. 11 illustrates another alternative embodiment of the example operational flow of FIG. 9

FIG. 12 illustrates a further embodiment of the example operation of FIG. 9;

FIG. 13 illustrates another embodiment of the example operational flow of FIG. 9;

FIG. 14 illustrates a further embodiment of the example operational flow of FIG. 9.

FIG. 15 illustrates an example environment that includes the animal, a cross-sectional view of an example final dosage form for administering the medicament to the animal, and the example stimulation source operable to emit a stimulus;

FIG. 16 illustrates an example environment that illustrates a final dosage form having a release element implemented by a characteristic response of a particle or a polymer to the stimulus;

FIG. 17 illustrates an example environment that includes an article;

FIG. 18 illustrates an example operational flow modifying a medicament availability characteristic of a final dosage form;

FIG. 19 illustrates an example final dosage form for administering a medicament to an animal;

FIG. 20 illustrates an example environment that includes a final dosage form 1502 configurable to administer a medicament to the animal;

FIG. 21 illustrates an example environment depicting retention and release states of particle or polymeric material (depicted as a hydrogel) responsive to an ex vivo stimulus;

FIG. 22 illustrates an example environment that includes an article of manufacture;

FIG. 23 illustrates an example environment that includes a final dosage form for administering the medicament to the animal;

FIG. 24 illustrates an example environment that includes a final dosage form and an operational flow;

FIG. 25 illustrates alternative embodiments of the activation operation of FIG. 24;

FIG. 26 illustrates an example embodiment of a final dosage form for administering a medicament; and

FIG. 27 illustrates an example system 2100 in which embodiments may be implemented.

### DETAILED DESCRIPTION

In the following detailed description, reference is made to the accompanying drawings, which form a part hereof. In the drawings, similar symbols typically identify similar components, unless context dictates otherwise. The illustrated embodiments described in the detailed description, drawings, and claims are not meant to be limiting. Other embodiments may be utilized, and other changes may be made, without departing from the spirit or scope of the subject matter presented here.

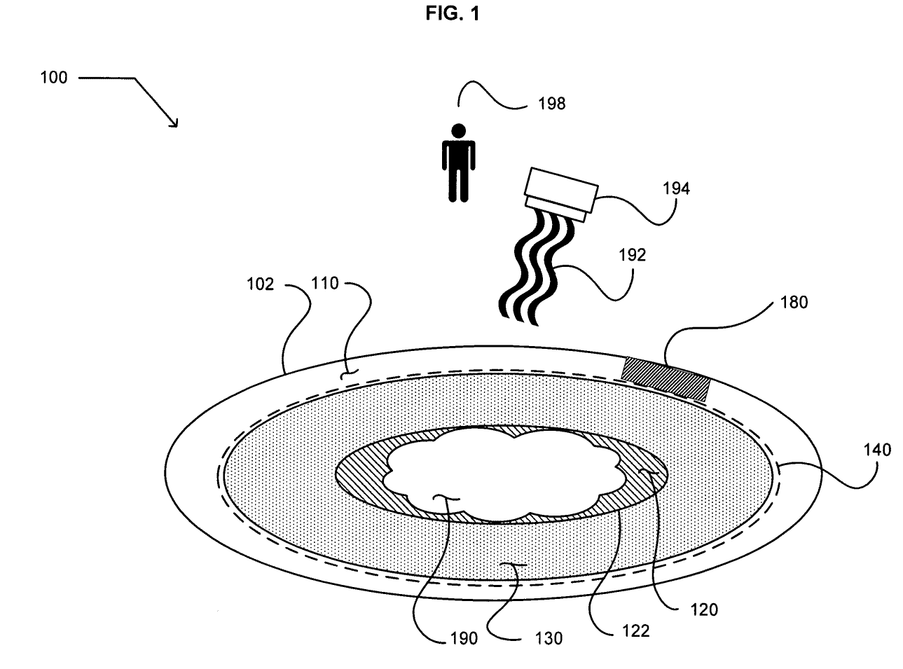

FIG. 1 illustrates an environment 100 that includes an animal 198, a cross-sectional view of an example final dosage form 102 for administering a medicament 190 to an animal, such as the animal 198, and an example stimulus source 194 configured to emit a stimulus 192. In an embodiment, the final dosage form includes a dosage form having completed a manufacturing or production process. In an embodiment, the final dosage form includes a product, finished tablet, or capsule ready for distribution to a hospital, pharmacy, or retail store for individualizing to a particular animal, such as the animal 198. In an embodiment, the final dosage form includes a tablet shape, a spherical shape, or an ellipsoidal shape. In an embodiment, the final dosage form includes a structure or a particle carryable or transportable by a liquid or other fluid carrier.

In an embodiment, the animal 198 includes any living being capable of voluntary movement and possessing specialized sense organs. In an embodiment, the animal includes a human. In an embodiment, the animal includes a mammal. In an embodiment, administering, administration, or administer the medicament to the animal includes give or apply the medicament 190 to the animal. In an embodiment, administering the medicament to the animal includes dispensing the medicament to the animal. In an embodiment, administering the medicament to the animal includes delivering the medicament to the animal. In an embodiment, administering the medicament to the animal includes directly or indirectly injecting the medicament to the animal. In an embodiment, administering the medicament to the animal includes applying the medicament to the animal. In an embodiment, administering the medicament to the animal includes providing the medicament to the animal.

The final dosage form 102 includes an outer layer 110, a release element 130, and a chamber 120. The release element 130 is configured in a first medicament-release state and modifiable ex vivo to a second medicament-release state by an exposure to a stimulus. For example, the stimulus may include the stimulus 192. The chamber 120 includes a chamber wall 122, which is substantially within the outer layer 110, and is configured to carry the medicament 190. In an embodiment, the final dosage form 102 includes an intermediate outer layer (not shown) with the release element interposed between the outer layer and the intermediate outer layer, and the chamber is substantially within the intermediate outer layer (not shown).

In an embodiment, the outer layer 110 of the final dosage form 102 includes an outer layer of at least one of a tablet, capsule, particle, or solid final dosage form. In an embodiment, the outer layer 110 includes an outer peripheral layer. FIG. 1 illustrates an example embodiment where the outer layer 110 includes an outer layer around the chamber wall 122 and the release element 130. In an embodiment, the outer layer 110 is configured for administration to the animal 198 by at least one of an oral, enteral, inhalation, or implant route. In an embodiment, an enteral route includes a rectal route, or a vaginal route, such as by a suppository. In an embodiment, the outer layer is configured for administration to the animal by at least one of parenteral, nasal, auditory canal, pulmonary, topical, or subdermal route.

In an embodiment, the outer layer 110 includes an outer layer configured to release the medicament in an in vivo environment of the animal. In an embodiment, the outer layer 110 includes an outer surface. In an embodiment, the outer layer includes an outer surface of a biocompatible medicament administration vehicle or transport.

In an embodiment, the outer layer 110 of the final dosage form 102 includes an erodible outer layer. Formulations of erodible dosage forms are known in the art. In an embodiment, the erodible outer layer includes an erodible outer layer that is at least one of soluble, permeable, or disintegrable within the animal 198. In an embodiment, the erodible outer layer includes an erodible outer layer having at least a portion that is at least one of soluble, permeable, or disintegrable in response to an acidic environment within the animal. In an embodiment, the erodible outer layer includes an erodible outer layer having at least a portion that is at least one of soluble, permeable, or disintegrable in response to a pH neutral or a basic environment within the animal.

In an embodiment, the outer layer 110 of the final dosage form 102 includes an outer portion of a plurality of particles. Examples of such a particle include one or more of hydrogels, microspheres, polymeric microspheres, and nanoparticles as described in Lin et al., Hydrogels in controlled release formulations: Network design and mathematical modeling, ADVANCED DRUG DELIVERY REVIEWS 58 (2006) (1379-1408). In an embodiment, the outer layer 110 of the final dosage form 102 includes an outer portion of an aggregation of molecules. An embodiment includes an outer layer 110 configured to allow an in vivo discharge of at least a portion of the medicament 190 from the chamber 120 after an exposure of the release element 130 to the stimulus 192. An embodiment includes an outer layer 110 configured in cooperation with the release element 130 to allow an in vivo discharge of at least a portion of the medicament 190 from the chamber 120 after an exposure of the release element 130 to the stimulus. In an embodiment, the outer layer 110 includes an outer layer of at least a portion of the release element. In an embodiment, the release element forms the outer layer. An embodiment includes an outer layer configured to contain the medicament until the final dosage form is administered into the animal.

In an embodiment of the release element 130, the first medicament-release state is configured to retard medicament release in vivo and the second medicament-release state is configured to allow medicament release in vivo. In an embodiment of the release element 130, the first medicament-release state is configured to allow medicament release in vivo and the second medicament-release state is configured to retard medicament release in vivo.

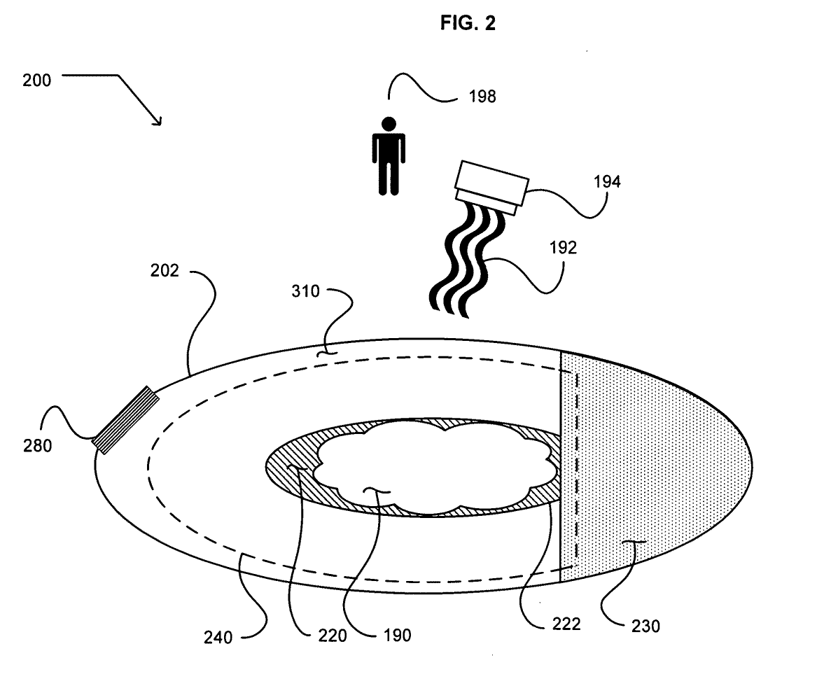

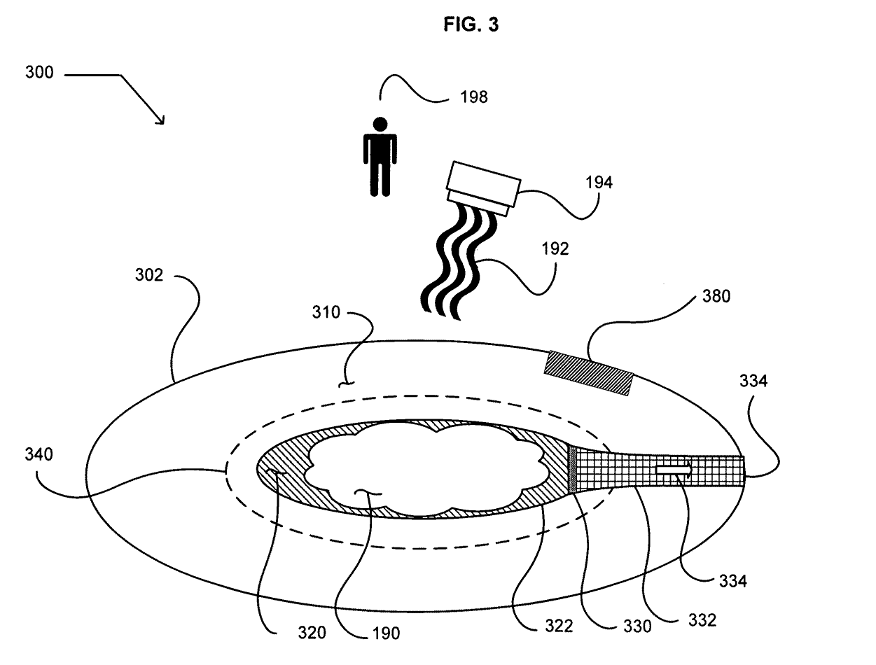

FIG. 1 illustrates a release element 130 disposed within the outer layer 110. In an embodiment, the release element includes a release element that is at least partially disposed within the outer layer, or a release element that is not disposed within the outer layer. For example, FIG. 2 infra, illustrates an example of a final dosage form 202 that includes a release element 230 that is not disposed within the outer layer 210. FIG. 3, infra, illustrates an example of a final dosage form 302 that includes a release element 330 disposed at least partially within the outer layer 310.

Returning to FIG. 1, in an embodiment, a release element 130 may be configured in a first medicament-release state and modifiable ex vivo to a second medicament-release state by an exposure to the stimulus 192. An embodiment includes a release element configured in a first medicament-release state and reconfigurable to a second medicament-release state by an exposure to the stimulus.

In an embodiment, the release element 130 includes a release element configured in a first medicament-release state and modifiable ex vivo to a second medicament-release state by an exposure to a non-ionizing radiation, illustrated as the stimulus 192. In an embodiment, the release element 130 is configured in a first medicament-release state and modifiable ex vivo to a second medicament-release state by an exposure to an electromagnetic radiation, illustrated as the stimulus 192. In an embodiment, the release element 130 is configured in a first medicament-release state and modifiable ex vivo to a second medicament-release state by an exposure to a light radiation, also illustrated as the stimulus 192. For example, light radiation may include at least one of the spectrum of ultraviolet (UV), visible light, /or infrared (IR).

In an embodiment, the release element 130 includes, but is not limited to, at least one of a poly(vinyl alcohol), gel, gel matrix, hydrogel, or azopolymer membrane. For example, a poly(vinyl alcohol) is described in (S. P. Vijayalakshmi, et al., Photodegradation of poly(vinyl alcohol) under UV and pulsed-laser irradiation in aqueous solution, JOURNAL OF APPLIED POLYMER SCIENCE, Vol. 102, No. 2, 958-966, 2006). For example, photo responsive polymers, including using an azopolymer with laser holography to generate the gated layer, are described in (J. Kyoo Lee, et. al., Photo-Triggering of the Membrane Gates in Photo-Responsive Polymer for Drug Release, ENGINEERING IN MEDICINE AND BIOLOGY SOCIETY, (27th Annual International Conference) 2005 Pages: 5069-5072 (2005). In an embodiment, the release element includes a photo-labile bond between a molecule of the medicament 190 and a bioactivity inhibiting molecule that is configured in a first medicament-release state and modifiable ex vivo to a second medicament-release state by an exposure of the labile bond to the stimulus. Examples of such a photo-labile bond are described in M. Scwarcznski, et al., Development of first photo responsive prodrug of paclitaxel, 16 BIOORGANIC & MEDICAL CHEMISTRY LETTERS, Issue 17 4492-4496 (September 2006): Epub 27 Jun. 2006. Scwarcznski, et al., describe synthesization of a prodrug of paclitaxel which has a coumarin derivative conjugated to the amino acid moiety of isotaxel (O-acyl isoform of paclitaxel). The prodrug was selectively converted to isotaxel by visible light irradiation (430 nm) with the cleavage of coumarin. Finally, paclitaxel was released by subsequent spontaneous O—N intramolecular acyl migration.

In addition, the release element may include at least one of an additional appropriate photodegradable /or biocompatible barrier forming material.

In an embodiment, the release element 130 includes a release element configured in a first medicament-release state and modifiable ex vivo to a second medicament-release state by an exposure to an energetic stimulus, also illustrated as stimulus 192. In an embodiment, an energetic stimulus may include at least one of a mechanical stimulus, a non-ionizing radiation stimulus, an ionizing radiation stimulus, a chemical stimulus, an acoustic stimulus, an ultrasound stimulus, a radio wave stimulus, a microwave stimulus, a light wave stimulus, or a thermal stimulus.

In an embodiment, the release element 130 is configured in a first medicament-release state and modifiable ex vivo to a second medicament-release state by an exposure to at least one of terahertz radiation, microwave radiation, and radio wave radiation, also illustrated as the stimulus 192. For example, radio wave radiation may include, for example, at least one of ultra-high frequency radio waves (UHF), very high frequency radio waves (VHF), radio frequency (RF), or extremely low frequency (ELF) radio waves. In an embodiment, the release element 130 includes at least one of a foil, gold foil, a liposome, wax, dielectric/wax composite. An example of a microwave responsive liposome is described in U.S. Pat. No. 4,801,459 to R. Liburdy. An example of a microwave responsive material, including a wax and a wax/dielectric composite, is described in United States Patent Application Publication No. 2005/0191708 to R. Saul, et al. In an embodiment, the release element is configured in a first medicament-release state and modifiable ex vivo to a second medicament-release state by an exposure to a magnetic stimulus. In an embodiment, the release element is configured in a first medicament-release state and modifiable ex vivo to a second medicament-release state by an exposure to an electric field stimulus.

In an embodiment, the release element is configured in a first medicament-release state and modifiable ex vivo to a second medicament-release state by an exposure to a chemical stimulus (not shown). For example, a chemical stimulus may include at least one of a stimulus based on pH change, enzymatic exposure or catalysis. In an embodiment, a chemical stimulus may include a stimulus operable to release or reverse a cooperative or a reversible molecular binding, or a stimulus operable to form an irreversible binding.

In an embodiment, the release element 130 is configured in a first medicament-release state and modifiable ex vivo to a second medicament-release state by an exposure to a mechanical agitation stimulus (not shown). For example, a mechanical agitation stimulus may include a shaking or spinning to rupture a membrane or a seal or a foil. In an embodiment, a release element is configured in a first medicament-release state and modifiable ex vivo to a second medicament-release state by an ex vivo exposure to a mechanical stimulus (not shown). For example, a mechanical stimulus may include shaking a piercing member against a foil release element. In an embodiment, the release element is configured in a first medicament-release state and modifiable ex vivo to a second medicament-release state by an ex vivo exposure to the stimulus, the release element including a mechanically activatable structure (not shown). For example, the mechanically activatable structure may include a foil or a pressure-rupturable membrane, or a heat-activatable structure.

In an embodiment, the release element 130 is permeated, dissolved, or disintegrated in response to the stimulus. In an embodiment, a release element is changed such that it is permeated, dissolved, or disintegrated in response to an in vivo environment of the animal 198 where it would not have been so before exposure to the stimulus. In an embodiment, a release element is changed such that it forms a barrier, or is impermeable, solid, or integral in response to the exposure to the stimulus where it would not have been so before the exposure to the stimulus.

In an embodiment, the release element 130 is configured in a first medicament-release state and modifiable ex vivo to a second medicament-release state by an exposure to at least one of a thermal, acoustic stimulus and ultrasound. Examples of an acoustically active release element formed by conjugating liposomes and microbubbles are described in A. Kheirolomoom, et al., Acoustically-active microbubbles conjugated to liposomes: Characterization of a proposed drug delivery vehicle, 118 J CONTROL RELEASE, Issue 3, April 23; 118(3):275-284. Epub 2006 Dec. 23.

In an embodiment, the release element 130 includes a release element configured in a first medicament-release state and modifiable ex vivo to a second medicament-release state by an ex vivo exposure to at least one of an activation stimulus, or an actuation stimulus. In an embodiment, the release element is configured in a first medicament-release state and modifiable ex vivo to a second medicament-release state by an exposure to a de-activation stimulus.

In an embodiment, the release element 130 includes a release element configured in a first medicament-release state and modifiable ex vivo to a second medicament-release state by an exposure to an ultrasound stimulus. For example, the release element may include at least one of liposomes, lipid microspheres, microbubbles, lipospheres, or liposomes responsive to an ultrasound stimulus, which are described in U.S. Pat. No. 6,416,740 to Unger. In an embodiment, the release element includes at least one of polyanhidrides, polyglycolides, polyactides, poly(vinyl acetate), poly(glycolic acid), poly(ethylene), poly(lactic acid), or chitosan. An example of ultrasound-responsive polymer is described in J. Kost, et al., Ultrasound-enhanced polymer degradation and release of incorporated substances, 86 PROCEEDINGS OF THE NATIONAL ACADEMY OF SCIENCES OF THE USA, 7663-7666 (1989). In this article, Kost describes up to a 5-fold reversible increase in degradation rate and up to 20-fold reversible increase in release rate of incorporated molecules were observed with biodegradable polyanhydrides, polyglycolides, and polylactides. This article also describes up to a 10-fold reversible increase in release rate of incorporated molecules within nonerodible ethylene/vinyl acetate copolymer were also observed. The release rate increased in proportion to the intensity of ultrasound. Temperature and mixing were relatively unimportant in effecting enhanced polymer degradation, whereas cavitation appeared to play a significant role. Another example of ultrasound-responsive polymer is described in J. Kost, et al., Ultrasonically controlled polymeric drug delivery, Makromolekulare Chemie 19 MACROMOLECULAR SYMPOSIA 275-285 (1988). In this article, Kost describes investigation of polymers that include lactic acid polymer, glycolic acid polymer, ethylene copolymer, vinyl acetate copolymer. An example of ultrasound-responsive chitosan is described in M. Tsaih, et al., Effect of the degree of deacetylation of chitosan on the kinetics of ultrasonic degradation of chitosan; 90 JOURNAL OF APPLIED POLYMER SCIENCE 3526-3531 (2003).

In an embodiment, the release element 130 includes at least one of polymeric micelle, liposomes, lipid microsomes, polymeric microsphere, nanoparticles, cyclodextrin, gel, gel matrix, hydrogel, or cellulose. Examples of polymeric micelles are described in U.S. Pat. No. 7,229,973 to Bae, et al. Bae describes polymeric micelles including mixed micelles containing poly(L-histidine)-poly(ethylene glycol) block copolymer and poly(L-lactic acid)-poly(ethylene glycol) block copolymer. Examples of polymer microspheres are described in U.S. Pat. No. 5,718,921 to Mathiowitz, et al. Mathiowitz describes polymer microspheres built using polyanhydrides, polyorthoesters, polylactic acid polymers, and combinations thereof. Examples of cyclodextrin are described in U.S. Pat. No. 7,270,808 to Cheng, et al., titled “Cyclodextrin-based polymers for therapeutics delivery.” Examples of hydrogels are described in Lin et al., Hydrogels in controlled release formulations: Network design and mathematical modeling, ADVANCED DRUG DELIVERY REVIEWS 58 (2006) 1379-1408). Examples of cellulose are described in U.S. Pat. No. 6,821,531 to Kumar.

In an embodiment, the release element 130 includes a release element enclosing the chamber 120, configured in a first medicament-release state, and modifiable ex vivo to a second medicament-release state by an exposure to a stimulus, illustrated as the stimulus 192. For example, FIG. 1 illustrates an embodiment where the outer layer 110 has a spherical shape, the chamber may have similar nested spherical shape, and the release element having a spherical shape and surrounding the chamber. However, nothing in this document expresses or implies a required similarity of shape among one or more of the chamber, the release element, or the outer layer. For example, an embodiment may include a liposome forming the release element and functionally defining a chamber.

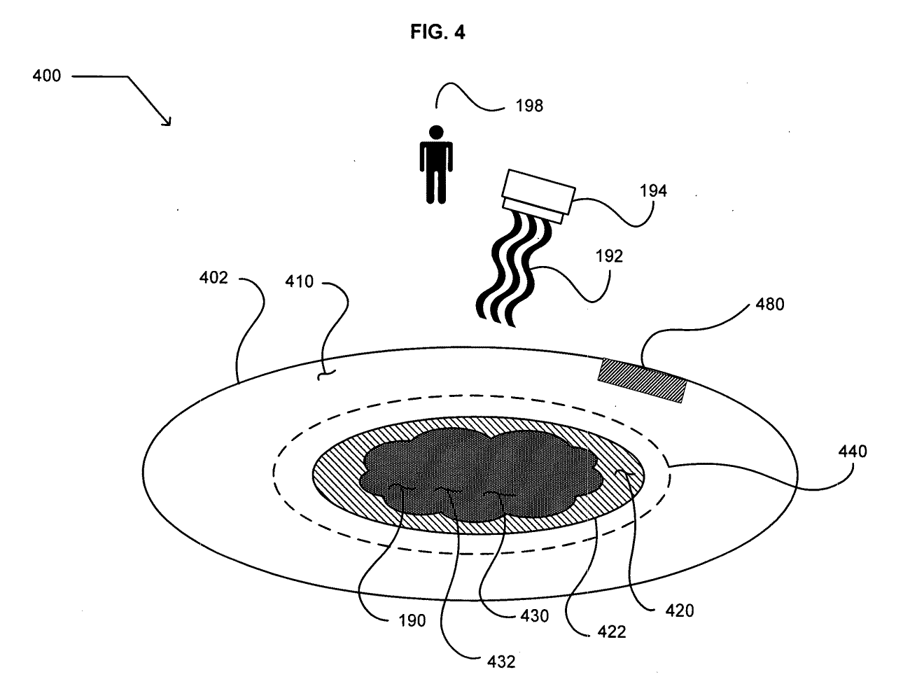

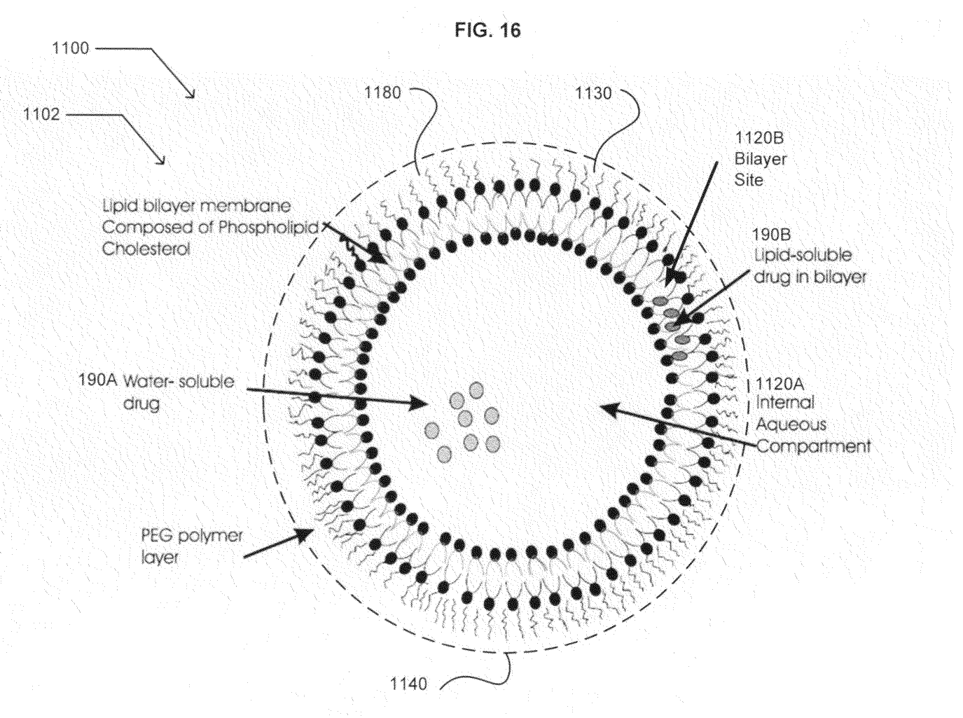

In an embodiment, the release element 130 includes or defines a release element encapsulating the chamber. In an embodiment, the release element includes a release element encapsulating the medicament 190 in cooperation with the chamber wall 122, configured in a first medicament-release state, and modifiable ex vivo to a second medicament-release state by an exposure to the stimulus. For example, FIG. 2, infra, illustrates a release element 230 encapsulating a medicament 190 in cooperation with a chamber 220 as expressed or defined by a chamber wall 222. In an embodiment, the release element includes a release element obstructing an aperture of the chamber. For example, FIG. 3, infra, illustrates a release element 330 in cooperation with a chamber 320 as expressed by a chamber wall 322 obstructing an aperture 332 of the chamber and preventing a discharge of a medicament 190 along a fluid communication path 336 In an embodiment, the release element includes at least two particles each collectively or respectively forming a chamber carrying a respective instance of the medicament. For example, FIG. 4, infra, illustrates a release element 430 that includes at a particle 432 forming a chamber carrying an instance of the medicament 190. An example of the particle 432 is additionally described in conjunction with FIGS. 16, 20, and 23, and respective corresponding particle or polymeric material 1180, 1580, and 1680. The particle is configured in a first medicament-release state, and modifiable ex vivo to a second medicament-release state by an exposure of the at least two particles to the stimulus. For example, the particle may include at least one of hydrogels, liposomes, or dendrimers configured to carry the medicament in an association with their pores, interstitial cavities, structural interstices, bonds, or amorphous cavities.

In an embodiment, the release element includes a labile bond between a molecule of the medicament and a bioactivity inhibiting molecule configured in a first medicament-release state and modifiable ex vivo to a second medicament-release state by an exposure of the labile bond to the stimulus (not shown).

Referring again to FIG. 1, in an embodiment, the release element 130 is configured in a first medicament-release state and modifiable ex vivo to a second medicament-release state by an exposure to the stimulus, and configured to contain the medicament 190 at least until the final dosage form 102 is administered into the animal 198.

FIG. 1 illustrates an embodiment having the chamber 120 formed within the outer layer 110 and configured to carry the medicament 190. In an embodiment, the chamber 120 is substantially defined within the outer layer and configured to carry the medicament until released by the release element. For example, FIG. 2 illustrates an embodiment that includes the chamber 220 substantially defined within the outer layer 210 and configured to carry the medicament 190 until released by the release element 230. FIG. 3 illustrates an embodiment that includes the chamber 320 substantially defined within the outer layer 310 and configured to carry the medicament 190 until released by the release element 330.

In an embodiment (not shown), the release element and chamber both may be formed by a particle, such as a liposome, or a hydrogel. In such embodiment, the chamber includes at least one chamber substantially within the outer layer of the particle and configured to carry the medicament.

In an embodiment, the chamber 120 includes a chamber configured to confine the medicament 190 in cooperation with the release element 130. In an embodiment (not shown), the chamber includes at least one chamber configured to confine the medicament in a structural cooperation with the release element. In an embodiment (not shown), the chamber is configured to initially carry the medicament. The chamber is also configured to release at least a portion of the medicament upon at least one of a reconfiguration, bursting, puncture, permeation, dissolution, and disintegration of the release element 130.

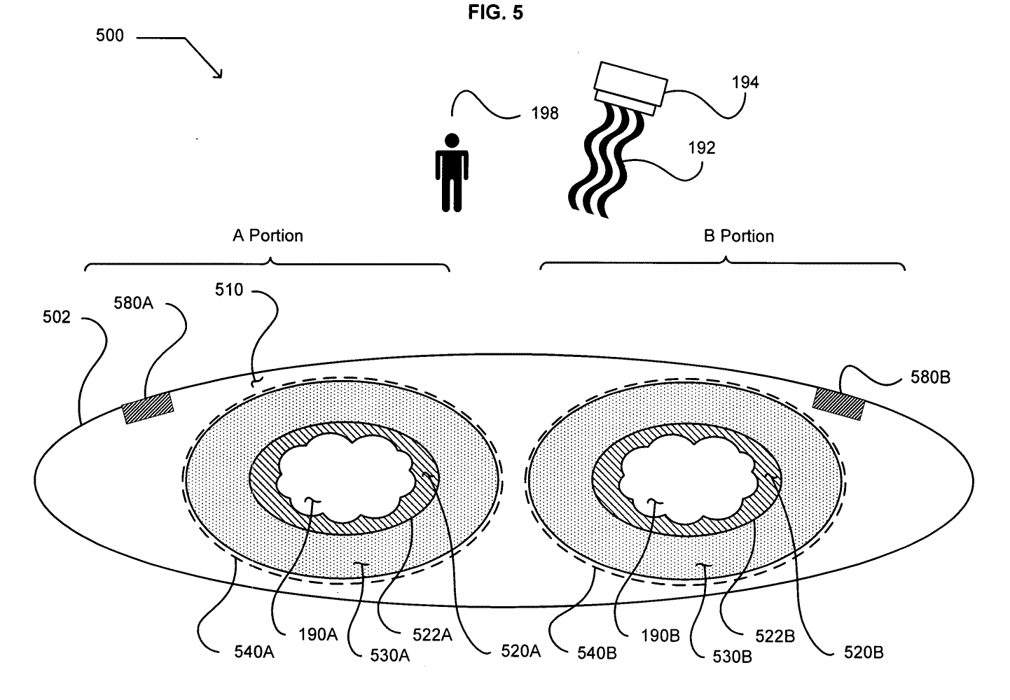

In an embodiment (not shown), the chamber 120 includes a first chamber configured to carry a first medicament and a second chamber configured to carry a second medicament. An example of the first chamber configured to carry a first medicament and the second chamber configured to carry a second medicament is described in conjunction with FIG. 5 and chamber 520A and chamber 520B. In an embodiment (not shown), the chamber includes a first chamber configured to carry a first constituent of the medicament and a second chamber configured to carry a second constituent of the medicament. In an embodiment (not shown), the chamber includes a first chamber configured to carry a first reactant of the medicament and a second chamber configured to carry a second reactant of the medicament. In an embodiment (not shown), a combination of the first reactant and the second reactant in response to an ex vivo exposure of the release element initiates a chemical activation or a synthesis of the medicament and a physical releasability of the medicament. In an embodiment (not shown), a combination of the first reactant and the second reactant in response to an ex vivo exposure of the release element initiates a chemical activation or a synthesis of the medicament but does not provide a physical releasability of the medicament. In an embodiment, the resulting medicament product can be released in vivo through the release characteristics of the outer layer. Alternatively, the physical releasability of the medicament may occur by another ex vivo exposure of the dosage form to a stimulus, such as the stimulus 192.

In an embodiment shown in FIG. 1, the final dosage form 102 includes a containment element 140 retaining the medicament 190 within the final dosage form until the dosage form is administered to the animal 198. The containment element can be used in situations where the medicament is a liquid or other material that is prone to seepage or discharge through the outer layer. In embodiment, the containment element may include a separate structure, such as a film or coating, retaining the medicament. Such a containment element 140 may form an exterior layer over the outer layer 110, or may form a layer interposed between the outer layer 110 and the chamber 120. In an embodiment, the containment element 140 may inhibit a discharge of the medicament 190 from the final dosage form 102 prior to its introduction into the animal 198, without regard to whether the release element is in its first medicament-release state or its second medicament-release state. In an embodiment, the containment element 140 includes a containment element 140 retaining the medicament 190 within the final dosage form 102 until the final dosage form 102 is exposed to an in vivo environment in the animal 198, and to modulate a release of at least a portion of the medicament 190 in vivo upon administration of the final dosage form 102 to the animal 198. In an embodiment, the containment element may be formed by a combination of the outer layer 110 and the release element 130.

In an embodiment, the containment element 140 includes a containment layer configured to encompass the medicament 190 within the final dosage form 102 until the final dosage form is administered to the animal 198. For example, the containment element 140 may include a coating covering the outer layer 110 of the final dosage form 102, such as an enteric coating configured to prevent a release of the medicament from the final dosage form until the final dosage form is administered to the animal. In another example, the containment element 140 may include a coating covering the release element 130 of the final dosage form 102. In an embodiment, the containment element includes a containment envelope retaining the medicament within the final dosage form until the dosage form is administered to the animal.

In an embodiment, the containment element 140 includes an enteric coating. The enteric coating may include gelatin or cellulose encapsulation. In an embodiment, the containment element includes a hydroxypropyl methylcellulose acetate succinate (HPMCAS) based coating or a methacrylic acid copolymer based coating, for example such as described in U.S. Pat. No. 7,138,143 to Mukai, et al. In an embodiment, the containment element includes a polymer coating, such as an acidic group-containing (meth)acrylate copolymer, shellac, HPMCP (hydroxypropylmethylcellulose phthalate), CAP (cellulose acetate phthalate), HIPMC-AS (hydroxypropylmethylcellulose acetate succinate) or polyvinyl acetate phthalate, for example such as described in U.S. Pat. No. 6,887,492 to Kay, et al. In an embodiment, the containment element includes a polymer coating of a (meth)acrylate copolymer comprising free-radical polymerized C.sub.1- to C.sub.4-alkyl esters of acrylic or methacrylic acid and (meth)acrylate monomers with a quaternary anunonium group in the alkyl radical, a (meth)acrylate copolymer of 20 to 40% by weight of polymerized ethyl acrylate and 60 to 80% by weight of polymerized methyl methacrylate, ethylcellulose or polyvinyl acetate. For example, as described in U.S. Pat. No. 6,897,205 to Beckert et al. In an embodiment, the containment element includes a cellulose acetate phthalate polymer coating material, for example, as described in U.S. Pat. No. 5,686,106 to Kelm, et al. In an embodiment, the containment element includes a cellulose acetate phthalate; cellulose acetate trimellitate; hydroxypropyl methylcellulose phthalate; hydroxypropyl methylcellulose acetate succinate; polyvinyl acetate phthalate; poly(methacrylic acid, methyl methacrylate) 1:1; or poly(methacrylic acid, ethyl acrylate) 1:1; and compatible mixtures thereof. In another embodiment, the containment element includes a poly(methacrylic acid, methyl methacrylate) 1:2, or a mixture of poly(methacrylic acid, methyl methacrylate) 1:1 and poly(methacrylic acid, methyl methacrylate) 1:2 in a ratio of about 1:10 to about 1:2. For example, as described in U.S. Pat. No. 5,686,105 to Kelm, et al.

In an embodiment illustrated in FIG. 2 infra, the containment element 240 includes a containment element 240 configured to prevent a release of the medicament 190 from the final dosage form 202 until the final dosage form 202 is introduced into the animal 198.

Returning to FIG. 1, in an embodiment, the medicament 190 includes at least one of an agent, treatment agent, drug, prodrug, therapeutic, nutraceutical, medication, vitamin, nutritional supplement, medicine, remedy, medicinal substance, or cosmetic. In an embodiment, the medicament includes a first reactant of the medicament and a second reactant of the medicament. In an embodiment, the medicament includes at least one prodrug and optionally an activating-enzyme of the prodrug. In an embodiment, the chamber includes a first chamber configured to carry a prodrug, and a second chamber configured to carry an activating enzyme of the prodrug.

In an embodiment, the final dosage form 102 may further include a indicator element 180 configured to indicate an exposure of the release element 130 to the stimulus 192. In an embodiment, the indicator element 180 includes an indicator element 180 configured to optically indicate an exposure of the release element to the stimulus 192 by at least one of dielectric, a conductivity, or ultrasonic profile responsive to an exposure of the release element to the stimulus. The indicator element 180 including, for example, at least one of 4-keto-bacteriorhodopsin films, cinnamylidene acetyl chloride, α-methylcinnamylidene acetyl chloride, α,γ-dimethylcinnamylidene acetyl chloride, α-phenylcinnamylidene acetyl chloride, α-phenoxycinnamylidene acetyl chloride, and cyanocinnamylidene acetyl chloride, leuco dye-serum albumin complexes, azo dyes, or poly(ethylene glycol). Examples of bacteriorhodopsin films are described in A. Druzhko, et al., 4-Keto-bacteriorhodopsin films as a promising photochromic and electrochromic biological material, BIOSYSTEMS. 1995; 35 (2-3): 129-32. Examples of hydrophilic photosensitive polymers are described in U.S. Pat. No. 5,990,193 to Russell, et al. Examples of photosensitive compositions for detection of radiation in the ultraviolet wavelength, including leuco dye-serum albumin complexes, are described in U.S. Pat. No. 4,466,941 to Cerami, et al. Examples of using azo dye for an indicator is described in U.S. Pat. No. 5,679,442. Examples of poly(ethylene glycol) are described in U.S. Pat. No. 5,990,193 to Russell, et al., and in Zhong, et al., Photodegradation Behavior of Polycaprolactone-Poly(ethylene glycol) Block Copolymer, Vol. 10, No. 4 CHINESE CHEMICAL LETTERS 327-330 (1999).

In an embodiment depicted in FIG. 1, the indicator element 180 includes an electronically-detectable indicator element 180 configured to indicate an exposure of the release element 130 to the stimulus 192. For example, the electronically-detectable indicator element 180 may include a substance, material, or device having a conductive property that makes an electronically-detectable change in response to an exposure to the stimulus 192. An example of such substance, material, or device includes a shape memory alloy switch that responds to heat described in U.S. Pat. No. 5,410,290 to Cho. Other examples of such substances, materials, or devices include a material that polymerizes in the presence of an ultrasound and changes a conductive property in response, such as the ultrasonic polymerization of methyl methacrylate described in U.S. Pat. No. 5,466,722 to Stoffer, et al., the heat or UV radiation triggered polymerization of acrylamide, or the microwave triggered polymerization of trimethylene carbonate. Another example of such substances, materials, or devices include the use of bistable compounds whose conductivity changes based upon exposure to electromagnetic radiation as described in U.S. Pat. No. 7,175,961 to Beck, et al. Another example includes a metal film or foil degradable by microwaves to release the medication whose state can degradation detected electrically.

In an embodiment, the indicator element 180 includes an electronically-detectable indicator element 180 configured to indicate an exposure of the release element 130 to the stimulus 192. For example, the electronically-detectable indicator element 180 may include a dielectric element having a property that makes an electronically-detectable change in response to an exposure to the stimulus 192. An example of such a dielectric element may include a one-time programmable memory cell described in U.S. Pat. No. 7,256,446, to Hu, et al., or a switch comprising microelectromechanical elements described in U.S. Pat. No. 7,336,474 to Lerche, et al.

In an example, the electronically-detectable indicator element 180 may include an element having a permittivity that makes an electronically-detectable change in response to an exposure of the release element to the stimulus 192. An example of such an element having a permittivity may include photonic crystals whose permittivity changes through the addition of photonic and/or electrical energy as described in U.S. Pat. No. 6,859,304 to Miller, et al.

In another example, the electronically-detectable indicator element 180 may include an element having an ultrasonic profile that makes an ultrasound-discernable change in response to an exposure of the release element to the stimulus 192. An example of an element having an ultrasonic profile that includes a polymer monitorable using the continuous wave ultrasonic process monitor is described in U.S. Pat. No. 7,017,412 to Thomas, et al. Another example of an element having an ultrasonic profile that includes a polymer monitorable using the apparatus for degree on doneness is described in U.S. Pat. No. 7,191,698 to Bond, et al. A further example of an element having an ultrasonic profile that includes a degradable metal film or metal foil.

In another example, the electronically-detectable indicator element 180 may include a carrier, admixture, diluent, or excipient having a property that makes an ultrasound-discernable change in response to an exposure of the release element to the stimulus 192. For example, an admixture may include a phase change material (PCM) as an inert filler and having a property that makes an ultrasound-discernable change in response to an exposure of the release element to ultrasound. Examples of such PCMs include polyvinyl alcohol (PVA)-stearic acid (SA) and polyvinyl chloride (PVC)-stearic acid (SA). An example of Polymer-stearic acid blend is described in Ahmet Sari, et al., Polymer-stearic acid blends as form-stable phase change material for thermal energy storage, 64 JOURNAL OF SCIENTIFIC & INDUSTRIAL RESEARCH, at pp. 991-996 (December 2005). Other examples are described in United States Patent Application No. 2007/0249753 to Lin, et al. (polyether fatty-acid ester (polyethylene glycol or polytetramethylene glycol base polymer), and U.S. Pat. No. 5,565,132 to Salyer (Addition of microwave absorber to make PCM materials sensitive to microwaves). Ultrasonic detection or discernment of phase changes in a PCM may be implemented using techniques described by A. W. Aziz, & S. N. Lawandy, Ultrasonic detection of segmental relaxations in thermoplastic polyurethanes, 31 JOURNAL OF APPLIED POLYMER SCIENCE 1585 (Issue 6, 2003) or S. L. Morton, Ultrasonic cure monitoring of photoresist during pre-exposure bake process, ULTRASONICS SYMPOSIUM, 1997. PROCEEDINGS., 1997 IEEE Volume 1, at 837-840 (October 1997).

The indicator element 180 (as enumerated in FIG. 1) can be made biocompatible so as to not cause an adverse reaction in the animal. Biocompatibility can be achieved through the use of a biocompatible material or through the use of a minimal amount of material so that any adverse reaction to the indicator element 180 is minimized.

FIG. 2 illustrates an environment 200 that includes the animal 198, a cross-sectional view of an example final dosage form 202 for administering the medicament 190 to the animal, and the example stimulation source 194 operable to emit the stimulus 192. In an embodiment, the final dosage form includes a dosage form having completed a manufacturing or production process. In an embodiment, the final dosage form includes a product, finished tablet, or capsule ready for distribution to a hospital, pharmacy, or retail store for individualizing to a particular animal, such as the animal 198. In an embodiment, the final dosage form may include a tablet shape, a spherical shape, or an ellipsoidal shape. In an embodiment, the final dosage form may include a structure or a particle carryable or transportable by a liquid or other fluid carrier.

The final dosage form 202 includes an outer layer 210, the release element 230, and the chamber 220 as expressed or defined by the chamber wall 222. The release element is configured in a first medicament-release state and modifiable ex vivo to a second medicament-release state by an exposure to a stimulus. For example, the stimulus may include the stimulus 192. The chamber includes a chamber wall 222, is substantially within the outer layer, and is configured to carry the medicament 190. In an embodiment, the final dosage form may include an indicator element 280. In an embodiment, the final dosage form may include a containment element 240.

The environment 200 illustrates an embodiment where the release element 230 encompasses the medicament 190 in cooperation with the chamber 220 as expressed or defined by the chamber wall 222. The outer layer 210 and the release element 230 are cooperatively retaining the medicament 190 if the release-element is in a first medicament-release state and allow an in vivo discharge of at least a portion of the medicament from the chamber if the release-element is in a second medicament release state. In an embodiment, the release element may include at least one of a poly(vinyl alcohol), gel, gel matrix, hydrogel, and azopolymer photo or light modifiable substance as described above. In an embodiment, the release element may include at least one of a polyanhidride, polyglycolide, polyactide, poly(vinyl acetate), poly(glycolic acid), poly (ethylene), poly(lactic acid), chitosan, or an acoustic or ultrasound-modifiable substance as described above. For example, when the first medicament-release state is configured to retard medicament release and the second medicament-release state is configured to allow medicament release in vivo, the release element when configured in the first medicament-release state will retard medicament release from the final dosage form upon administration of the final dosage form into the animal. For example, in a first medicament release state, the release element is impermeable to the environment outside the final dosage form, and impermeable to the medicament in the chamber. Following exposure to an appropriately configured stimulus, the release element achieves a second medicament release state that is, for example, permeable to the medicament. The second medicament release state may include, for example, a state where the release element dissolves or dissipates upon exposure to an aqueous environment, gastric juices or a certain pH environment.

FIG. 3 illustrates a non-limiting environment 300 that includes the animal 198, a cross-sectional view of an example final dosage form 302 for administering the medicament 190 to the animal, and the example stimulation source 194 operable to emit the stimulus 192. In an embodiment, the final dosage form includes a dosage form having completed a manufacturing or production process. In an embodiment, the final dosage form includes a product, finished tablet, or capsule ready for distribution to a hospital, pharmacy, or retail store for individualizing to a particular animal, such as the animal 198. In an embodiment, the final dosage form may include a tablet shape, a spherical shape, or an ellipsoidal shape. In an embodiment, the final dosage form may include a structure or a particle carryable or transportable by a liquid or other fluid carrier.

The final dosage form 302 includes an outer layer 310, a chamber 320, and a release element 330. The final dosage form also includes a release passageway 332 configured to provide a medicament communication pathway between the chamber and the environment through an aperture 334 in the outer layer. The release element is configured in a first medicament-release state and modifiable ex vivo to a second medicament-release state by an exposure to a stimulus. For example, the stimulus may include the stimulus 192. The chamber includes a chamber wall 322, is substantially within the outer layer, and is configured to carry the medicament 190. In an embodiment, the final dosage form may include an indicator element 380. In an embodiment, the final dosage form may include a containment element 340.

FIG. 3 illustrates a non-limiting embodiment wherein an embodiment of the final dosage form 302 includes the release element 330 retaining the medicament 190 in cooperation with the chamber 320 as expressed by the chamber wall 322. The outer layer 310 and the release-element 330 are cooperatively retaining the medicament 190 if the release-element is in one medicament-release state and allowing an in vivo discharge of at least a portion of the medicament from the chamber if the release-element is in another medicament release state. When the release-element is in a state that releases the medicament, the medicament may discharge or flow along the fluid communication path 336 expressed or defined at least in part by the release passageway 332.

In an embodiment, the release element may include at least one of a poly(vinyl alcohol), gel, gel matrix, hydrogel, and azopolymer photo or light modifiable substance as described above. In an embodiment, the release element may include at least one of a foil, gold foil, wax, or dielectric/wax composite microwave modifiable substance. In an embodiment of this example, the release element may include at least one of a polyanhidride, polyglycolide, polyactide, poly(vinyl acetate), poly(glycolic acid), poly(ethylene), poly(lactic acid), chitosan, or an acoustic or ultrasound-modifiable substance as described above. For example, when the first medicament-release state is configured to retard medicament release and the second medicament-release state is configured to allow medicament release in vivo, the release element when configured in the first medicament-release state will retard medicament release from the release passageway 332 and the aperture 334 of the final dosage form upon administration of the final dosage form into the animal.

FIG. 4 illustrates an environment 400 that includes the animal 198, a cross-sectional view of an example final dosage form 402 for administering the medicament 190 to the animal, and the example stimulation source 194 operable to emit the stimulus 192. In an embodiment, the final dosage form 402 includes a dosage form having completed a manufacturing or production process. In an embodiment, the final dosage form 402 includes a product, finished tablet, or capsule ready for distribution to a hospital, pharmacy, or retail store for individualizing to a particular animal, such as the animal 198. In an embodiment, the final dosage form 402 may include a tablet shape, a spherical shape, or an ellipsoidal shape. In an embodiment, the final dosage form 402 may include a structure or a particle carryable or transportable by a liquid or other fluid carrier.

The final dosage form 402 includes an outer layer 410, a chamber 420, and a release element 430. The release element is configured in a first medicament-release state and modifiable ex vivo to a second medicament-release state by an exposure to a stimulus. For example, the stimulus may include the stimulus 192. The chamber includes a chamber wall 422, is substantially within the outer layer, and is configured to carry the medicament 190. In an embodiment, the final dosage form 402 may include an indicator element 480. In an embodiment, the final dosage form 402 may include a containment element 440.

In an embodiment, the chamber 420 includes a chamber substantially within the outer layer 410 and configured to carry the medicament 190. In an embodiment, the chamber encloses at least two structures within the chamber having respective subchambers configured to carry the medicament. For example, such at least two structures may include at least two pores, molecular structures having interstitial cavities, smaller chambers, molecular structure having interstices, or molecular structure having amorphous cavities. In an embodiment, the chamber may contain at least one of an absorbent, liposome, or hydrogel molecular structure which define respective chambers therein. For example, at least two particles may be located in a cavity, such as the chamber 120, and in themselves define a distributed chamber by an aggregation of their pores, interstitial cavities, smaller chambers, interstices of a molecular structure, or amorphous cavities. In another example, at least two microparticles may be throughout a carrier having an outer layer, each microparticle having an effective chamber. In an embodiment, the chamber is located substantially within the release element 430. In an embodiment, the distributed chamber is located substantially within the outer layer 410.

The final dosage form 402 may include a release element 430 that is associated with the medicament 190 in the chamber 420. In an embodiment, the release element 430 may include a carrier, admixture, diluent, or excipient configured in a first medicament-release state and modifiable ex vivo to a second medicament-release state by an ex vivo exposure to the stimulus 192. Particles of such a carrier, admixture, diluent, or excipient may be configured to retain or bind to particles of the medicament 190 and reduce its bioavailability if the release-element 430 is in a first medicament-release state, and release from or unbind particles of the medicament 190 and allow an in vivo discharge of at least a portion of the medicament 190 from the chamber 420 if the release-element 430 is in a second medicament release state.

In an embodiment, an instance of the final dosage form 402 may carry at least two particles, small particles, or microparticles that each include a portion that forms a release element 430 modifiable ex vivo by exposure to the stimulus 192, and a chamber (not shown). The chambers of the at least two particles, small particles, or microparticles each configured to carry a respective instance of the medicament, and collectively forming a distributed chamber. For example, the at least two particles, small particles, or microparticles may include hydrogels, liposomes, or dendrimers having pores, interstitial cavities, structural interstices, bonds, or amorphous cavities configurable to carry molecules of the medicament. The at least two particles, small particles, or microparticles are configured in a first medicament-release state and modifiable ex vivo to a second medicament-release state by an exposure of the at least two particles, small particles, or microparticles to the stimulus. For example, photosensitive hydrogel particles may carry the medicament. In an embodiment, microwave sensitive liposomes may carry the medicament. In an embodiment, the release element includes a labile bond between a molecule of the medicament and molecule of a bioactivity inhibiting molecule configured in a first medicament-release state and modifiable ex vivo to a second medicament-release state by an exposure of the labile bond to the stimulus (not illustrated).

FIG. 5 illustrates an environment 500 that includes an animal 198, a cross-sectional view of a final dosage form 502 for transporting a medicament to the animal. The medicament is illustrated as a first medicament 190A and second medicament 190B. In an embodiment, the final dosage form includes a dosage form having completed a manufacturing or production process. In an embodiment, the final dosage form 502 includes a product, finished tablet, or capsule ready for distribution to a hospital, pharmacy, or retail store for individualizing to a particular animal, such as the animal 198. In an embodiment, the final dosage form 502 may include a tablet shape, a spherical shape, or an ellipsoidal shape. In an embodiment, the final dosage form 502 may include a structure or a particle carryable or transportable by a liquid or other fluid carrier.

The final dosage form 502 includes an outer layer 510, and at least two dosage elements. The at least two dosage elements are illustrated as A Portion and B Portion, and by “A” and “B” after certain reference numbers in FIG. 5. The A Portion includes a chamber 520A, a release element 530A, and a medicament 190A. In an embodiment, the A Portion includes a containment element 540A. In an embodiment, the A Portion includes an indicator element 580A. The B Portion includes a chamber 520B, a release element 530B, and a medicament 190B. In an embodiment, the B Portion includes a containment element 540B. In an embodiment, the B Portion includes an indicator element 580B.

In an embodiment, the A Portion of the final dosage form 502 may be substantially similar to the chamber 120, the release element 130, the containment element 140, and the indicator element 180 of FIG. 1. In an embodiment, the A Portion may be substantially similar to the chamber 220, the release element 230, the containment element 240, and the indicator element 280 of FIG. 2. In an embodiment, the A Portion may be substantially similar to the chamber 320, the release element 330, the containment element 340, and the indicator element 380 of FIG. 3. In an embodiment, the A Portion may be substantially similar to the chamber 420, the release element 430, the containment element 440, and the indicator element 480 of FIG. 4. Similarly, the B Portion of the final dosage form 502 may be substantially similar to that described in conjunction with at least one of FIG. 1, FIG. 2, FIG. 3, or FIG. 4.

In an embodiment, the first medicament 190A and the second medicament 190B may be substantially similar instances of the same medicament. In an embodiment, the first medicament 190A and the second medicament 190B may be substantially similar instances of the same medicament, but in substantially differing dosage amounts. For example, the first medicament 190A may be a 50-milligram dose of a medicament and the second medicament 190B may be a 100-milligram dose of the same medicament. In an embodiment, the first medicament 190A and the second medicament 190B may be substantially similar instances of the same medicament, but in substantially differing dosage characteristics, such as a regular release formulation and a sustained release formulation. In an embodiment, the first medicament 190A and the second medicament 190B may be different medicaments.

In use, the A Portion and the B Portion of the final dosage form 502 may be individually or collectively exposed ex vivo to a stimulus, illustrated as the stimulus 192. For example, where the first medicament 190A is a 50-milligram dose of a medicament and the second medicament 190B is a 100-milligram dose of a same medicament, where the release element 530A and release element 530B are modifiable ex vivo by the same stimulus, such as microwave energy, and where the first medicament-release state is configured to retard medicament release in vivo and the second medicament-release state is configured to allow medicament release in vivo, irradiation of the A Portion with microwave energy will actuate the A Portion and make 50-milligrams of the medicament available upon administration of the final dosage form to the animal 198. Similarly, irradiation of the B Portion with microwave energy will actuate the B Portion and make 100-milligrams of the medicament available upon administration of the final dosage form to the animal. Further, irradiation of both the A Portion and the B Portion with microwave energy will actuate both Portions and make 150-milligrams of the medicament available upon administration of the final dosage form to the animal. In another example, the first medicament 190A is a 100-milligram dose of a first medicament and the second medicament 190B is a 100-milligram dose of a second medicament. Selective irradiation of the A Portion or the B Portion will make one or both of the medicaments bioavailable upon administration of the final dosage form to the animal. In a further example, the release element 530A is modifiable ex vivo by a first stimulus and the release element 530B is modifiable ex vivo by the second and different stimulus.

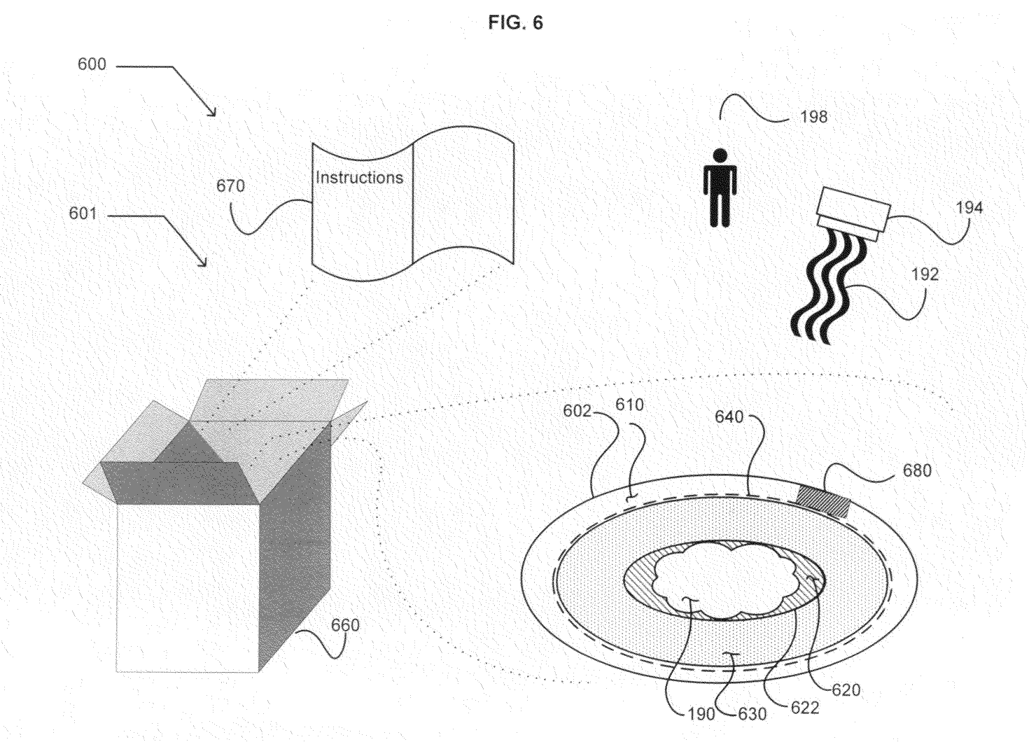

FIG. 6 illustrates an example environment 600 that includes an article of manufacture 601. The article of manufacture includes a package 660 containing a final dosage form 602 and providing an instruction 670. The final dosage form includes a medicament 190, an outer layer 610, a release element 630, and a chamber 620. The release element is configured in a first medicament-release state and modifiable ex vivo to a second medicament-release state by an exposure to an actuation-stimulus. The chamber lies substantially within the outer layer and is configured to carry the medicament. The instruction includes instruction for preparation of the final dosage form for an efficacious administration to an animal by an exposure of the release element of the final dosage form to the stimulus.

In an embodiment, the final dosage form 602 may be substantially similar to the final dosage form 102 of FIG. 1. In an embodiment, the final dosage form 602 may be substantially similar to the final dosage form 202 of FIG. 2. In an embodiment, the final dosage form 602 may be substantially similar to the final dosage form 302 of FIG. 3. In an embodiment, the final dosage form 602 may be substantially similar to the final dosage form 402 of FIG. 4. In an embodiment, the final dosage form 602 may be substantially similar to the final dosage form 502 of FIG. 5.

In an embodiment, the instruction 670 includes at least one of information indicating an actuation-stimulus type, an actuation-stimulus wavelength, an actuation-stimulus intensity, an actuation-stimulus duration, a spatial distribution of the stimulus relative to the final dosage form, a target-value for an exposure indicator, or a combination thereof. For example, the information indicating a spatial distribution of the stimulus relative to the final dosage form may include information corresponding to aiming the stimulus, such as toward a right hand portion, a center portion, or a left hand portion of the final dosage form. In an embodiment, the instruction includes an instruction presented by at least one of a label (not shown) on the package 660, an insert in the package, illustrated as the instruction 670, or an address to electronically published content (not shown). In an embodiment, the instruction includes instruction for preparation of the final dosage form for an efficacious administration to an animal by a human-initiated exposure of the release element of the final dosage form to the actuation-stimulus.

In an embodiment, the final dosage form 602 further includes a containment element 640 retaining the medicament within the final dosage form until the final dosage form is introduced into the animal. In an embodiment, the final dosage form includes an indicator element 680 configured to indicate an exposure of the release element to the stimulus. In an embodiment, the instruction 670 includes information indicating an expected value of the indicator element.

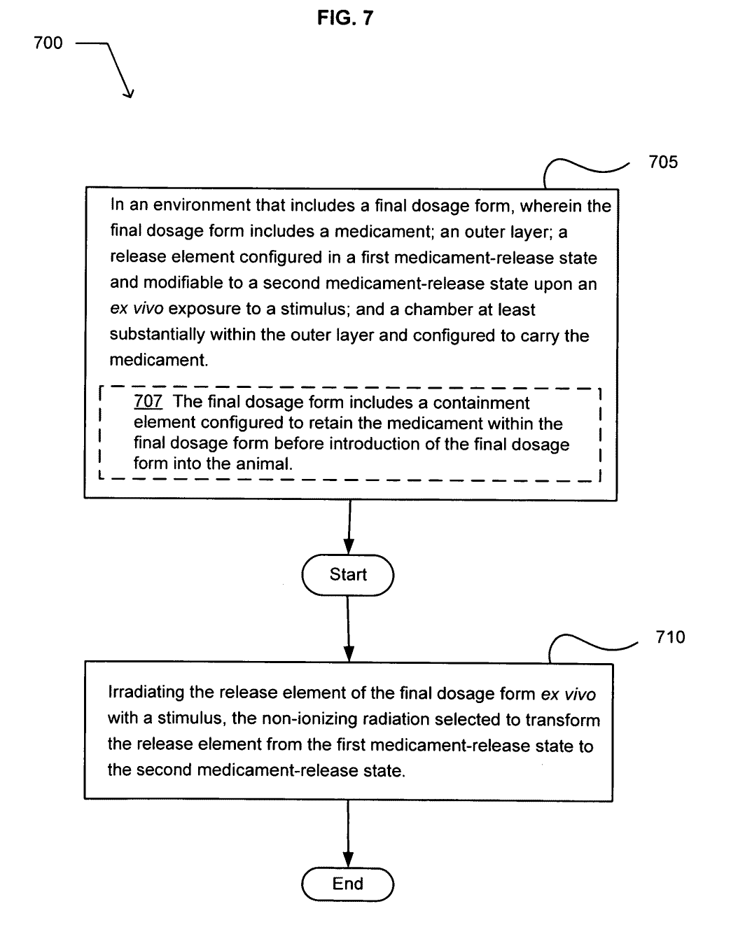

FIG. 7 illustrates an example operational flow 700 modulating a medicament-release characteristic of a final dosage form. A start operation occurs in an environment 705 that includes the final dosage form. The final dosage form includes a medicament, an outer layer, a release element configured in a first medicament-release state and modifiable ex vivo to a second medicament-release state by an exposure to the stimulus, and a chamber substantially within the outer layer and configured to carry the medicament. After the start operation, the operational flow includes an individualization operation 710. The individualization operation includes irradiating the release element of the final dosage form ex vivo with a non ionizing radiation, the non-ionizing radiation selected to transform the release element from the first medicament-release state to the second medicament-release state. For example, the irradiating the release element of the final dosage form ex vivo with the stimulus may occur in a hospital pharmacy, a retail pharmacy, a battlefield hospital, a veterinary facility, or other location dispensing medicaments. In another example, the irradiating a release element of the final dosage form ex vivo with the stimulus may occur in a persons home. The operational flow then proceeds to an end operation. In an alternative embodiment, the final dosage form further includes a containment element retaining the medicament within the final dosage form before introduction of the final dosage form into the animal.

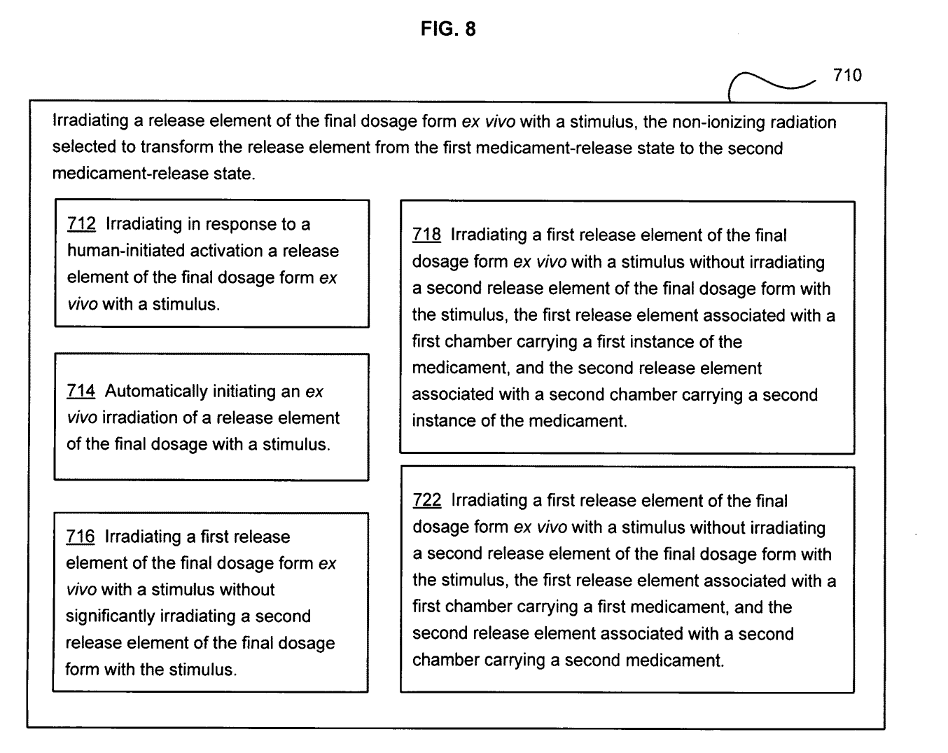

FIG. 8 illustrates an alternative embodiment of the operational flow 700 of FIG. 7. The individualization operation 710 may include at least one additional operation. The at least one additional operation may include at least one of an operation 712, an operation 714, an operation 716, an operation 718, or an operation 722. The operation 712 includes irradiating in response to a human-initiated activation a release element of the final dosage form ex vivo with a non-ionizing radiation. The operation 714 includes automatically initiating an ex vivo irradiation with a non-ionizing radiation a release element of the final dosage. The operation 716 includes irradiating a first release element of the final dosage form ex vivo with a non-ionizing radiation without irradiating a second release element of the final dosage form with the stimulus. The operation 718 includes irradiating a first release element of the final dosage form ex vivo with a non-ionizing radiation without irradiating a second release element of the final dosage form with the non-ionizing radiation. The first release element is associated with a first chamber carrying a first instance of the medicament, and the second release element is associated with a second chamber carrying a second instance of the medicament. The operation 722 includes irradiating a first release element of the final dosage form ex vivo with a non-ionizing radiation without irradiating a second release element of the final dosage form with the stimulus. The first release element is associated with a first chamber carrying a first medicament, and the second release element is associated with a second chamber carrying a second medicament.

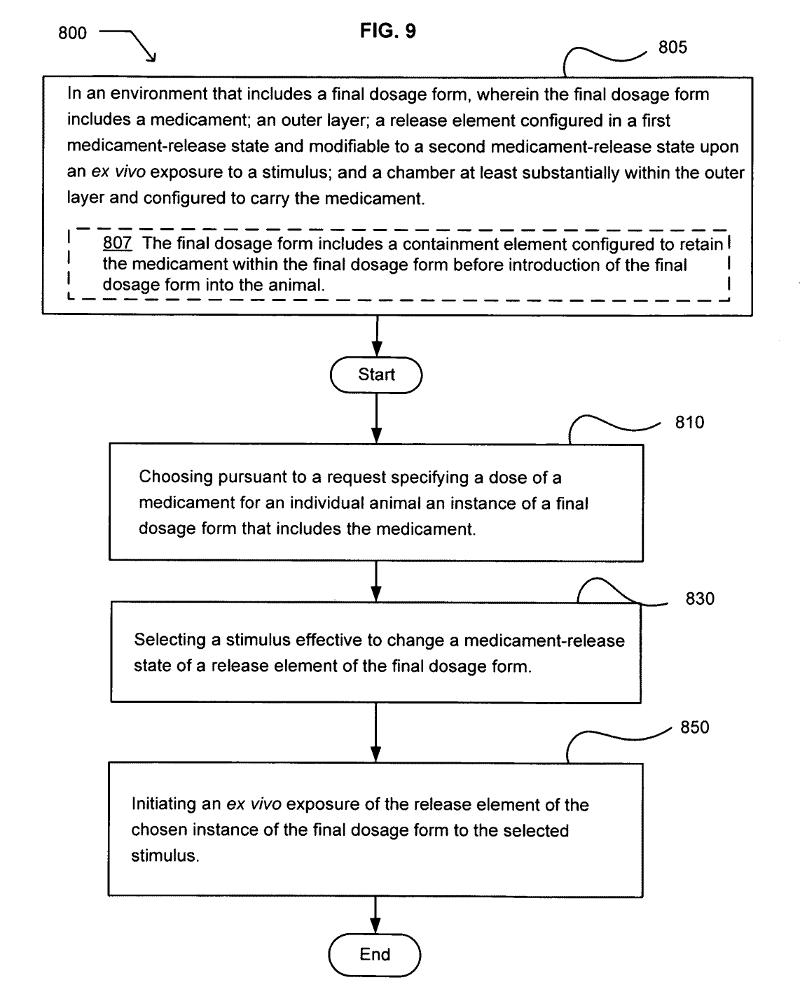

FIG. 9 illustrates an example operational flow 800 fulfilling a request specifying a dose of a medicament for an individual animal. A start operation occurs in an environment that includes a final dosage form. The final dosage form includes an outer layer, a release element configured in a first medicament-release state and changeable to a second medicament-release state by an exposure to the stimulus, a chamber substantially within the outer layer and configured to carry the medicament, and the medicament. In an alternative embodiment, the final dosage form further includes a containment element retaining the medicament within the final dosage form before introduction of the final dosage form into the animal. After the start operation, the operational flow includes a picking operation 810. The picking operation includes choosing pursuant to the request an instance of a final dosage form that includes the medicament. A decision operation 830 includes selecting a stimulus configured to change or transform a medicament-release state of a release element of the final dosage form. A customization operation 850 includes initiating an exposure of the release element of the chosen instance of the final dosage form to the selected stimulus. The operational flow then proceeds to an end operation.

In use of an embodiment, a person such as a pharmacist working in a pharmacy may receive a prescription specifying a dose of a medicament for a patient. A pharmacy typically may have available several different final dosage forms capable of administering the prescribed medicament dose. For example, the available different dosage forms may include at least one of the embodiments of final dosage forms illustrated in FIGS. 1-5. In a picking operation, the pharmacist chooses pursuant to the request an instance of a final dosage form that includes the medicament. In a decision operation, the pharmacist selects a stimulus effective to change a medicament-release state of a release element of the final dosage form. The pharmacist may select the stimulus after consulting with an instruction presented by at least one of a label on box containing the chosen instance of a final dosage form, a package insert in the box, or an address to electronically published content indicated on the label, or package insert. The pharmacist enters the selected stimulus setting for a stimulus emitter, such as the stimulus emitter 194 of FIG. 1. In a customization operation, the pharmacist initiates an exposure of the release element of the chosen instance of the final dosage form to the selected stimulus. The pharmacist may confirm exposure of the release element to the stimulus by referring to the indicator element. For example, the indicator element may change color in response to an exposure to the selected stimulus. If the prescription specifies multiple doses of the medicament for the patient, the pharmacist may repeat the above sequence until sufficient doses have customized. Alternatively, and if appropriate for the chosen final dosage forms, multiple instances of the final dosage form may be ex vivo exposed to the selected stimulus at one time.

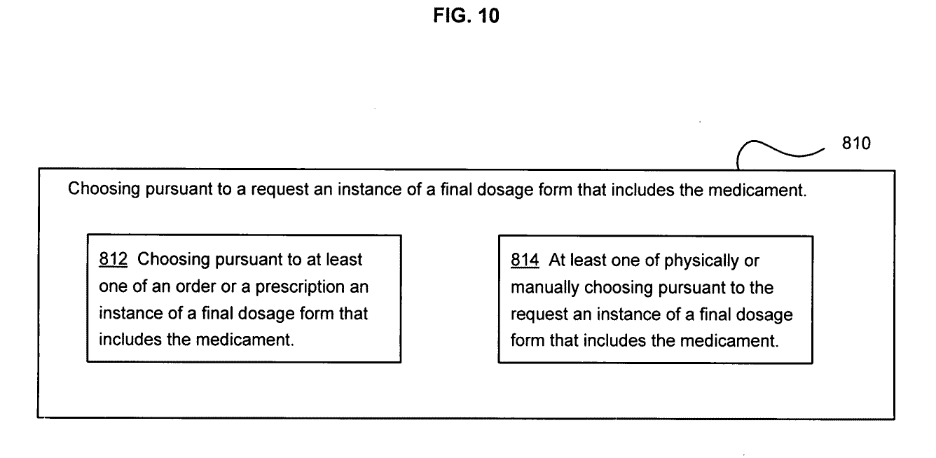

FIG. 10 illustrates an alternative embodiment of the example operational flow 800 of FIG. 9. The picking operation 810 may include at least one additional operation. The at least one additional operation may include an operation 812, or an operation 814. The operation 812 includes choosing pursuant to at least one of an order or a prescription an instance of a final dosage form that includes the medicament. The operation 814 includes at least one of physically or manually choosing pursuant to the request an instance of a final dosage form that includes the medicament.

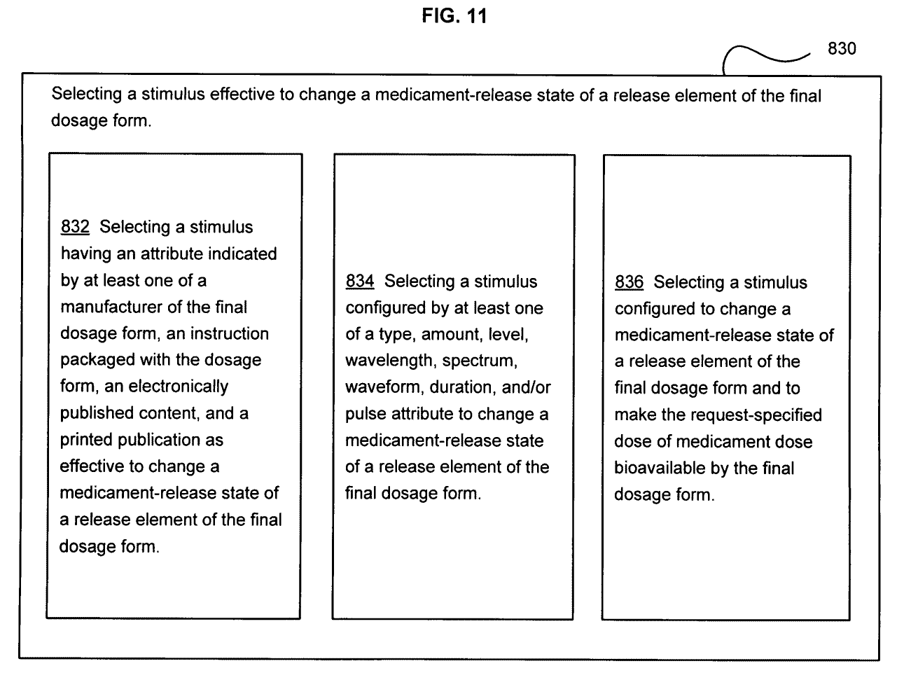

FIG. 11 illustrates another embodiment of the example operational flow 800 of FIG. 9. The decision operation 830 may include at least one additional operation. The at least one additional operation may include an operation 832, an operation 834, or an operation 836. The operation 832 includes selecting a stimulus having an attribute indicated by at least one of a manufacturer of the final dosage form, an instruction packaged with the dosage form, an electronically published content, and a printed publication as effective to change a medicament-release state of a release element of the final dosage form. For example, electronically published content may include a website maintained by the manufacturer of the final dosage form. In a further example, a printed publication may include a reference book, such as Physician's Desk Reference. The operation 834 includes selecting a stimulus configured by at least one of a type, amount, level, wavelength, spectrum, waveform, spatial distribution, duration, or pulse attribute to change a medicament-release state of a release element of the final dosage form. The operation 836 includes selecting a stimulus configured to change a medicament-release state of a release element of the final dosage form and to make the request-specified dose of medicament dose bioavailable by the final dosage form.

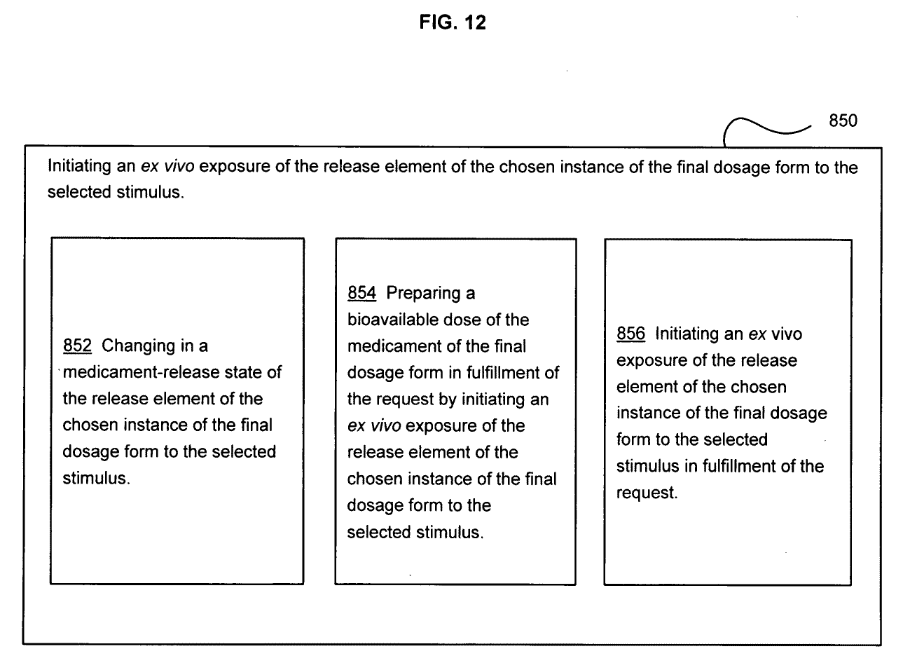

FIG. 12 illustrates an embodiment of the example operation 800 of FIG. 9. The customization operation 850 may include at least one additional operation. The at least one additional operation may include an operation 852, an operation 854, or an operation 856. The operation 852 includes changing a medicament-release state of the release element of the chosen instance of the final dosage form by initiating an exposure of the release element of the chosen instance of the final dosage form to the selected stimulus. The operation 854 includes preparing a bioavailable dose of the medicament of the final dosage form in fulfillment of the request by initiating an exposure of the release element of the chosen instance of the final dosage form to the selected stimulus. The operation 856 includes initiating an exposure of the release element of the chosen instance of the final dosage form to the selected stimulus in fulfillment of the request.

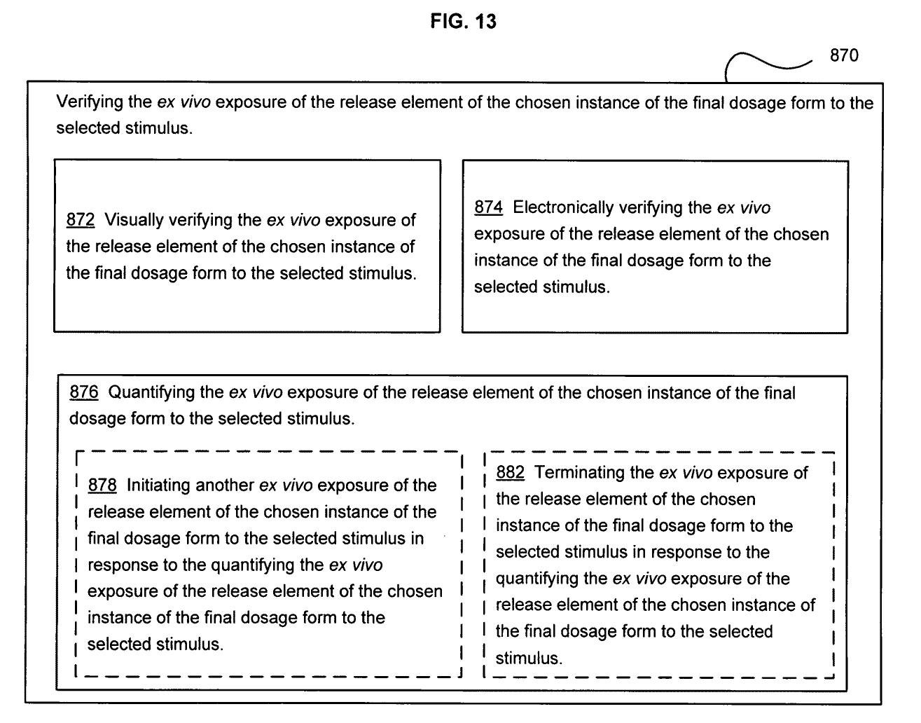

FIG. 13 illustrates an embodiment of the example operational flow 800 of FIG. 9. The operation 870 may include at least one additional operation. The at least one additional operation may include an operation 872, an operation 874, or an operation 876. The operation 870 may include at least one additional operation. The at least one additional operation may include an operation 872, an operation 874, or an operation 876. The operation 872 includes optically verifying the exposure of the release element of the chosen instance of the final dosage form to the selected stimulus. For example, optically verifying the exposure may be implemented using human vision, machine vision, or ultrasound techniques. The operation 874 includes electronically verifying the exposure of the release element of the chosen instance of the final dosage form to the selected stimulus. For example, electronically verifying the exposure of the release element may be implemented using a dielectric element having a property that makes an electronically discernable change in response to an exposure to the stimulus. The operation 876 includes quantifying the exposure of the release element of the chosen instance of the final dosage form to the selected stimulus. The operation 876 may include at least one additional operation. The at least one additional operation may include an operation 878, or an operation 882. The operation 878 includes initiating another exposure of the release element of the chosen instance of the final dosage form to the selected stimulus in response to the quantifying the exposure of the release element of the chosen instance of the final dosage form to the selected stimulus. The operation 882 includes terminating the exposure of the release element of the chosen instance of the final dosage form to the selected stimulus in response to the quantifying the exposure of the release element of the chosen instance of the final dosage form to the selected stimulus.

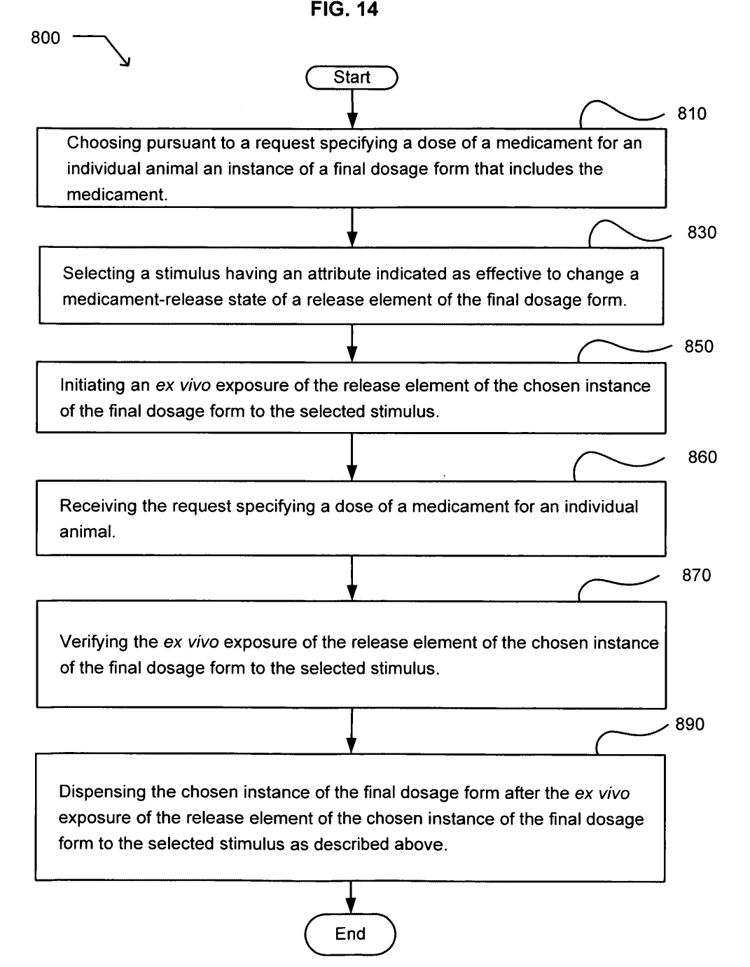

FIG. 14 illustrates an embodiment of the example operational flow 800 of FIG. 9. The operational flow 800 may include at least one additional operation. The at least one additional operation may include an operation 860, an operation 870, or an operation 890. The operation 860 includes receiving the request specifying a dose of a medicament for an individual animal. The operation 860 may include at least one additional operation. The at least one additional operation may include an operation 862, or an operation 864. The operation 862 (not shown) includes receiving the request specifying an efficacious medicament dose for an individual animal. The operation 864 (not shown) includes receiving the request specifying the final dosage form that includes the medicament for an individual animal.

The operation 870 includes verifying the exposure of the release element of the chosen instance of the final dosage form to the selected stimulus. The operation 890 includes dispensing the chosen instance of the final dosage form after the exposure of the release element of the chosen instance of the final dosage form to the selected stimulus as described above. The operation 890 may include at least one additional operation, such as an operation 892. The operation 892 (not shown) includes dispensing the ex vivo exposed instance of the final dosage form in a package bearing an identifier of the individual animal. For example, the identifier may include a name, or identification number of the animal.

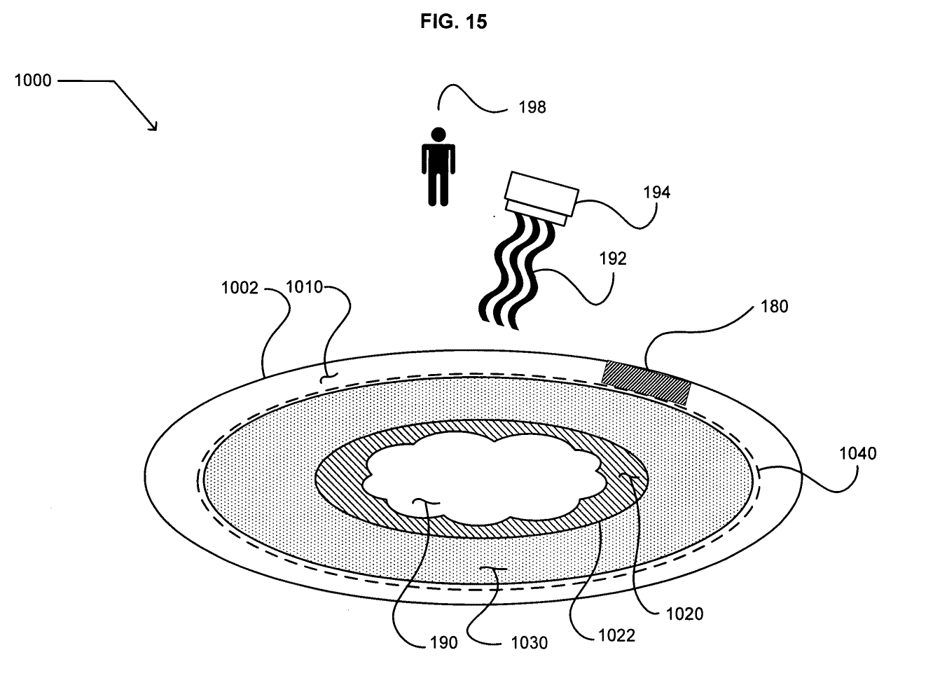

FIG. 15 illustrates an example environment 1000 that includes the animal 198, a cross-sectional view of an example final dosage form 1002 for administering the medicament 190 to the animal 198, and the example stimulation source 194 operable to emit a stimulus 192. In an embodiment, the final dosage form includes a dosage form having completed a manufacturing or production process. In an embodiment, the final dosage form includes a product, finished tablet, or capsule ready for distribution to a hospital, pharmacy, or retail store for individualizing for a particular animal. In an embodiment, the final dosage form includes a tablet shape, a spherical shape, or an ellipsoidal shape. In an embodiment, the final dosage form includes a structure, a particle, or a polymer that is carryable or transportable to the animal by a solid, cream, liquid, or fluid carrier.

The final dosage form 1002 includes an outer layer 1010, a release element 1030, and the medicament 190. The release element is configured in a medicament-holding state. The release element is modifiable ex vivo to a medicament-discharge state by an exposure to a stimulus, illustrated as the stimulus 192. In an embodiment, ex vivo includes outside the body of the animal. In an embodiment, ex vivo includes an environment outside or away from the body of the animal. In an embodiment, ex vivo includes outside a living organism, such as “in vitro.” In an embodiment, ex vivo includes an external or ambient environment.

In an embodiment, the stimulus 192 includes at least one of a non-ionizing radiation, an electromagnetic radiation, a magnetic field, an electric field, an energetic stimulus, or a chemical stimulus. In an embodiment, the stimulus includes at least one of a light radiation, terahertz radiation, microwave radiation, or radio wave radiation. In an embodiment, the stimulus includes at least one of a mechanically activatable structure, heat activatable structure, or pressure activatable structure. In an embodiment, the stimulus includes at least one of a thermal, acoustic, or ultrasound stimulus. In an embodiment, the stimulus includes at least one of an activation stimulus, or an actuation stimulus.

In an embodiment, the release element 1030 includes a release element configured in a medicament-holding state. In the medicament-holding state, the medicament 190 is substantially not bioavailable to the animal 198 if the final dosage form 1002 is administered to the animal. The release element is modifiable ex vivo to a medicament-discharge state by an exposure to the stimulus 192. In the medicament-discharge state, the medicament is substantially bioavailable to the animal if the final dosage form is administered to the animal. In an embodiment, substantially not bioavailable to the animal includes having no substantial therapeutic or adverse effect on the animal. In an embodiment, bioavailable to the animal includes the medicament being physiologically available, absorbable, transportable, usable, or utilizable by the animal. In an embodiment, bioavailable to the animal indicates that a portion of an administered dose of medicament reaches the systemic circulation. In an embodiment, not bioavailable to the animal includes the medicament being physiologically not available, not absorbable, not transportable, not usable, or not utilizable by the animal.

In an embodiment, the release element 1030 includes a release element configured in a medicament-holding state. In the medicament-holding state, the medicament 190 is insubstantially bioavailable if the final dosage form 1002 is administered to the animal 198. The release element is modifiable ex vivo to a medicament-discharge state by an exposure to the stimulus 192. In the medicament-discharge state, the medicament is substantially bioavailable if the final dosage form is administered to the animal. In an embodiment, the release element includes a release element configured in a medicament-holding state. In the medicament-holding state, the medicament is substantially bio-unavailable if the final dosage form is administered to the animal. The release element is modifiable ex vivo to a medicament-discharge state by an exposure to the stimulus wherein the medicament is substantially bioavailable if the final dosage form is administered to the animal. In an embodiment, bio-unavailable includes present but not usable by the animal.

In an embodiment, the release element includes a release element 1030 configured in a medicament-holding state. In the medicament-holding state, the medicament 190 has a substantially insignificant effect on the animal 198 if the final dosage form 1002 is administered to the animal. The release element is modifiable ex vivo to a medicament-discharge state by an exposure to the stimulus. In the medicament-discharge state, the medicament has a substantially significant effect on the animal if the final dosage form is administered to the animal. In an embodiment, the release element includes a release element configured in a medicament-withholding state, and modifiable ex vivo to a medicament-supplying state by an exposure to the stimulus.

In an embodiment, the release element 1030 includes a release element configured in a medicament-holding state and field-modifiable ex vivo to a medicament-discharge state by an exposure to the stimulus 192. For example, the release element may be field modified at a point of administration of the final dosage form, such as clinic or hospital, at a pharmacy such as when a pharmacist is filling a prescription that includes the final dosage form, or at a residence by a caregiver or by a person for whom the final dosage form is prescribed. In an embodiment, the release element includes a release element configured in a medicament-holding state and modifiable ex vivo post-manufacture to a medicament-discharge state by an exposure to the stimulus. In an embodiment, the release element includes a release element forming an imperforate barrier in a medicament-holding state. The release element is modifiable ex vivo by an exposure to the stimulus to form a perforate barrier in a medicament-discharge state. In an alternative embodiment, the release element is modifiable ex vivo by an exposure to the stimulus to form a perforate barrier in a medicament-discharge state to form at least one discharge pathway.

In an embodiment, the release element 1030 includes a release element configured in a medicament-holding state. The release element is modifiable ex vivo to a medicament-discharge state by an exposure to the stimulus 192, the stimulus including at least one of light or radio waves.

In an embodiment, the final dosage form 1002 includes a particle or polymer implemented release element. In an embodiment, a polymer may include an intelligent polymer having a changeable property that in one state admits or discharges at least one molecule of medicament and in another state engages or retains the at least one molecule of medicament. An intelligent polymer includes a polymer structurally responsive to an externally applied energy or stimulus. In an embodiment, “applied energy” includes both positive and negative energy values, i.e. supplying and removing energy. Examples of intelligent polymers are described in U.S. Pat. No. 7,104,517 to Derand, et al. In an embodiment, a particle may include a microsphere, polymeric microsphere, or nanoparticle.

FIG. 16 illustrates an example environment 1100 that illustrates a final dosage form 1102 having a release element 1130 implemented by a characteristic response of a particle or a polymeric material 1180 to a stimulus such as, the stimulus 192. An example of the particle or polymeric material is illustrated as a liposome. The final dosage form includes molecules of the medicament 190 carried by the particle or a polymeric material, again illustrated with respect to the liposome. Example water-soluble drug molecules 190A are illustrated as engaged, retained, or entrapped in an internal aqueous compartment site 1120A. Example lipid-soluble drug molecules 190B are illustrated as engaged, retained or entrapped in a bilayer site 1120B. The example environment 1100 illustrates the release element configured in a medicament-holding state with at least one molecule of the medicament engaged, retained or entrapped. In an embodiment, the release element has, for example by application of a stimulus, such as the stimulus 192, been changed into a state (not shown) that admits at least one molecule of the medicament, illustrated as the water-soluble drug molecules 190A or the lipid-soluble drug molecules 190B. The release element state is changed by withdrawal of the stimulus into a state (shown) that engages, retains or entraps the water-soluble drug molecules 190A or the lipid-soluble drug molecules 190B. Continuing this example, the release element is modifiable ex vivo to a medicament-discharge state by an exposure to a stimulus, such as the stimulus 192, which may be the same stimulus used to switch the release element and load the water-soluble drug molecules 190A or the lipid-soluble drug 190B into the liposome, or may be another stimulus 192. In an alternative of this example, a chemical stimulus may be used to load the at least one molecule in the liposome, and another stimulus, such as an electromagnetic wave used to modify the liposome to a medicament-discharge state.

The characteristic response of the particle or polymeric material to the stimulus 192 may include any characteristic response that releases an engaged, retained, or entrapped medicament 190 from the particle or polymeric material. For example, a characteristic response of a particular particle or polymeric material may include a releasing bursting, expanding, cleaving, or degradation of the particular particle or polymeric material in response to a microwave stimulus implements the release element 1130.

In an embodiment, the release element 1130 is configured in a medicament-holding state. The release element is modifiable ex vivo to a medicament-discharge state by an exposure to the stimulus 192. The release element including at least one of a gel, gel matrix, hydrogel fibrin, or a dendrimer. Examples hydrogels are described in Y. Qiu, et al, Environment-sensitive hydrogels for drug delivery, SCIENCEDIRECT (October 2001), citing Triggering in Drug Delivery Systems, 53 ADVANCED DRUG DELIVERY REVIEWS 321-339 (Issue 3, December 2001). Examples polymers and dendrimers are described in C. Henry, Drug Delivery, 80 CHEMICAL & ENGINEERING NEWS 39-47 (No. 34, Aug. 26, 2002) (The drugs are conjugated to the dendrimers using photocleavable or labile linkers, which can be made to release the drug using light or through acid cleavage). Examples of photo-labile, radio-labile, and enzyme-labile dendrimers are described in U.S. Pat. No. 6,471,968 to Baker, et al.; and in U.S. Pat. No. 7,078,461 to Tomalia, et al. In an embodiment, a particle or polymeric material having a characteristic responsive to an exposure to the stimulus include an intelligent or environmentally sensitive particle or polymeric material.

In an embodiment, the release element 1130 includes a particle (not specifically shown) configured in a medicament-holding state. The particle is modifiable ex vivo to a medicament-discharge state by an exposure to the stimulus 192. Examples of environmentally sensitive particles such as microspheres have been described previously herein. In an embodiment, the release element includes a polymer substance configured in a medicament-holding state. The polymer substance is modifiable ex vivo to a medicament-discharge state by an exposure to the stimulus. Examples of environmentally sensitive polymer substances have been described previously herein.

Returning to FIG. 15, in an embodiment, the release element 1030 includes a non-shape-memory material configured in a medicament-holding state. The non-shape-memory material is modifiable ex vivo to a medicament-discharge state by an exposure to the stimulus 192. In an embodiment, the release element includes a release element disposed at least partially within the outer layer 1010 and configured in a medicament-holding state. The release element is modifiable ex vivo to a medicament-discharge state by an exposure to the stimulus. In the medicament-discharge state the medicament 190 is substantially available for an in vivo release into the animal 198 if the final dosage form is administered to the animal while the release element is configured in the medicament-discharge state.

In an embodiment, the final dosage form 1002 further includes a chamber 1020 located substantially within the outer layer 1010 and carrying the medicament 190. In an embodiment, the chamber 1020 is substantially similar to the chamber 120 described in conjunction with FIG. 1. In an embodiment, the final dosage form further includes the indicator element 180 configured to indicate an exposure of the release element 1030 to the stimulus 192.

Another embodiment provides a final dosage form 1002 for administering the medicament 190 to an animal 198. This embodiment of the final dosage may be illustrated by reference to FIG. 15 and/or by reference to FIG. 16. With reference to an embodiment illustrated by FIG. 15, the final dosage form 1002 includes a release element 1030, a site 1020 carrying the medicament, the medicament, and a containment element 1040. The release element 1030 is configured in a medicament-holding state and modifiable ex vivo to a medicament-discharge state by an exposure to the stimulus 192. The containment element retains the medicament within the final dosage form until the final dosage form is administered to the animal. In an embodiment, the containment element may be substantially similar to the containment element 140 described in conjunction with FIG. 1.

With reference to an embodiment illustrated by FIG. 16, an embodiment of the final dosage form includes the final dosage form 1102 having a release element 1130 formed by a particle or polymer 1180, a site carrying the medicament 190 (depicted as the internal aqueous compartment site 1120A or as the bilayer site 1120B), the medicament. The release element is configured in a medicament-holding state and modifiable ex vivo to a medicament-discharge state by an exposure to the stimulus.

In an embodiment, the final dosage form 1102 includes a containment element 1140. The containment element retains the medicament within the final dosage form until the final dosage form is administered to the animal. In an embodiment, the containment element 1140 includes a particle or polymeric substance retaining the medicament 190 within the final dosage form 1102 until the final dosage form is administered to the animal 198 (not shown). For example, the containment element may include a gel, hydrogel, liposome microsphere, polymeric microsphere, dendrimer, or nanoparticle. In an embodiment, the containment element may be another particle or polymer that engages, retains, or entraps the particle or polymer in a containing manner (not shown). In an embodiment, the containment element may be substantially similar to the containment element 140 of the final dosage from 102 described in conjunction with FIG. 1. In an embodiment, the containment element may be substantially similar to the erodible outer layer 110 of the final dosage form 102 described in conjunction with FIG. 1. In an embodiment, the release element 1130 and the containment element are an at least a substantially same structure, for example a microsphere. In an embodiment, the release element and the containment element are substantially different structures, for example a microsphere containment element containing a dendrimer release element. In an embodiment, the containment element includes a carrier, excipient, diluent, or admixture retaining the medicament within the final dosage form until the final dosage form is administered to the animal.

In an embodiment, the site 1120 carrying the medicament 190 includes a chamber carrying the medicament. In an embodiment, the site carrying the medicament includes a region carrying the medicament. In an embodiment, the site carrying the medicament includes a binding site releasably carrying the medicament. In an embodiment, the site carrying the medicament includes the release element 1130 or a particle or polymeric material carrying the medicament. In an embodiment, the site carrying the medicament includes a binding site releasably carrying the medicament.

In an embodiment, the final dosage form includes an indicator element 180 configured to indicate an exposure of the release element to the stimulus. With reference to an embodiment illustrated by FIG. 15, the final dosage form 1002 includes an indicator element 180 configured to indicate an exposure of the release element 1030 to the stimulus 192. With reference to FIG. 16, the final dosage form 1102 includes an indicator element (not shown) configured to indicate an exposure of the release element 1130 to the stimulus 192.

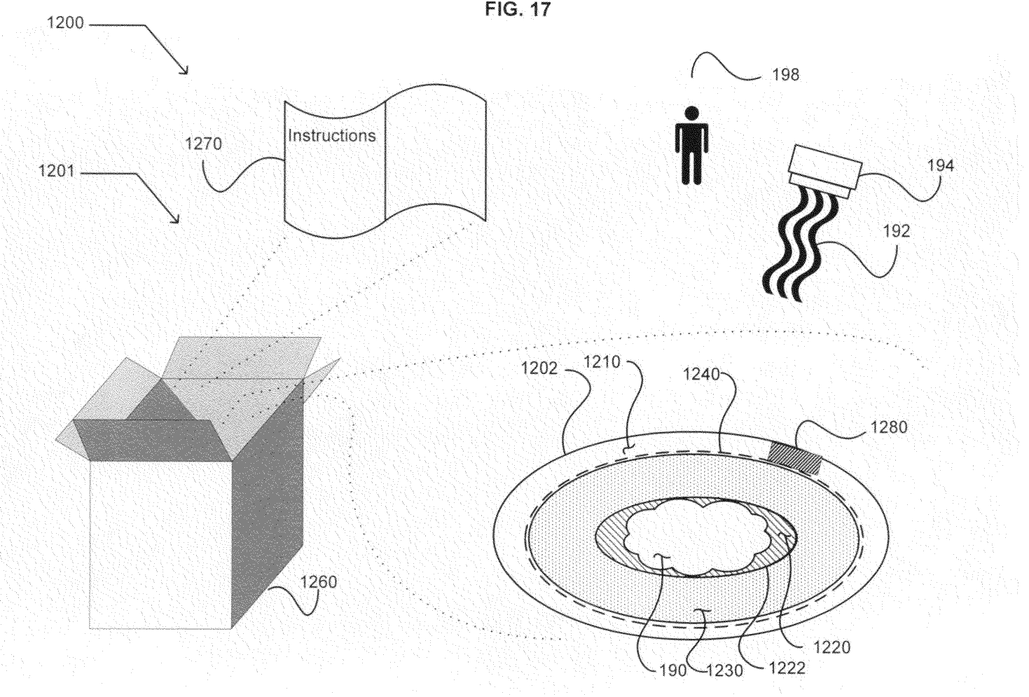

FIG. 17 illustrates an example environment 1200 that includes an article 1201. The article includes at least one final dosage form for administering a therapeutically effective amount of a medicament to an animal. In an embodiment, the final dosage form includes a final dosage 1202 that is substantially similar to the final dosage form 1002 described in conjunction with FIG. 15. In an embodiment, the final dosage form includes the final dosage form 1202 that is substantially similar to the final dosage form 1102 described in conjunction with FIG. 16 (not shown in FIG. 17). The final dosage form of FIG. 17 includes an outer layer 1210, a release element 1230, a site 1220 carrying the medicament 190. The release element includes a release element configured in a medicament-holding state. The release element is modifiable ex vivo to a medicament-discharge state by an exposure to the stimulus 192. The article also includes instructions 1270 for the exposure of the release element to the stimulus sufficient to modify the release element to the medicament-discharge state. In an embodiment, the instructions include instructions specifying the exposure of the release element to a human-initiated stimulus sufficient to modify the release element to the medicament-discharge state. In an embodiment, the instructions include instructions specifying the exposure of the release element to provide an automatically-initiated stimulus sufficient to modify the release element to the medicament-discharge state.

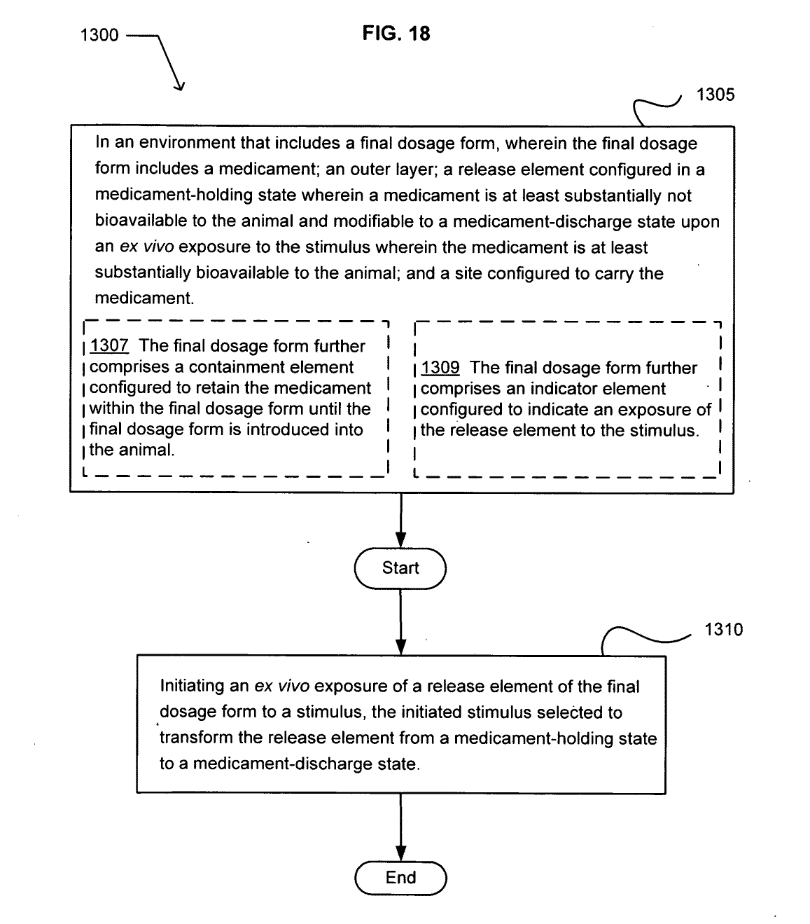

FIG. 18 illustrates an example operational flow 1300 modifying a medicament availability characteristic of a final dosage form. A start operation occurs in an environment 1305 that includes the final dosage form. The final dosage form includes an outer layer, a release element, a site carrying the medicament, and the medicament. The release element is configured in a medicament-holding state wherein a medicament is substantially not bioavailable to the animal, such as the animal 198. The release element is modifiable ex vivo to a medicament-discharge state by an exposure to the stimulus wherein the medicament is substantially bioavailable to the animal. In an embodiment, the final dosage form is substantially similar to the final dosage form 1002 described in conjunction with FIG. 15. In an embodiment, the final dosage form is substantially similar to the final dosage form 1102 described in conjunction with FIG. 16

After the start operation, the operational flow 1300 includes an activating operation 1310. The activating operation includes initiating an exposure of a release element of the final dosage form to a stimulus, such as the stimulus 192. The initiated stimulus is selected to transform the release element from a medicament-holding state to a medicament-discharge state. In an embodiment, the initiated stimulus includes an initiated stimulus having a parameter selected to transform the release element from a medicament-holding state to a medicament-discharge state. In an embodiment, the initiated stimulus includes an initiated stimulus having at least one of a stimulation characteristic or a spatial characteristic selected to transform the release element from a medicament-holding state to a medicament-discharge state. In an embodiment, the initiating an exposure of a release element of the final dosage form to a stimulus includes initiating a first exposure of a release element of the final dosage form to a stimulus. The initiated first stimulus is selected to transform the release element from a medicament-holding state to a medicament-discharge state. This embodiment further includes receiving an indication of the first exposure of the release element of the final dosage form to the stimulus, the indication generated in response to an indicator element of the final dosage form configured to indicate an exposure of the release element to the stimulus. This embodiment further includes initiating a second exposure of the release element of the final dosage form to the stimulus. The initiated second exposure stimulus is selected to further transform the release element from the medicament-holding state to the medicament-discharge state.

In an embodiment, the final dosage form includes a containment element retaining the medicament within the final dosage form until the final dosage form is introduced into the animal. In an embodiment, the final dosage form includes an indicator element configured to indicate an exposure of the release element to the stimulus.

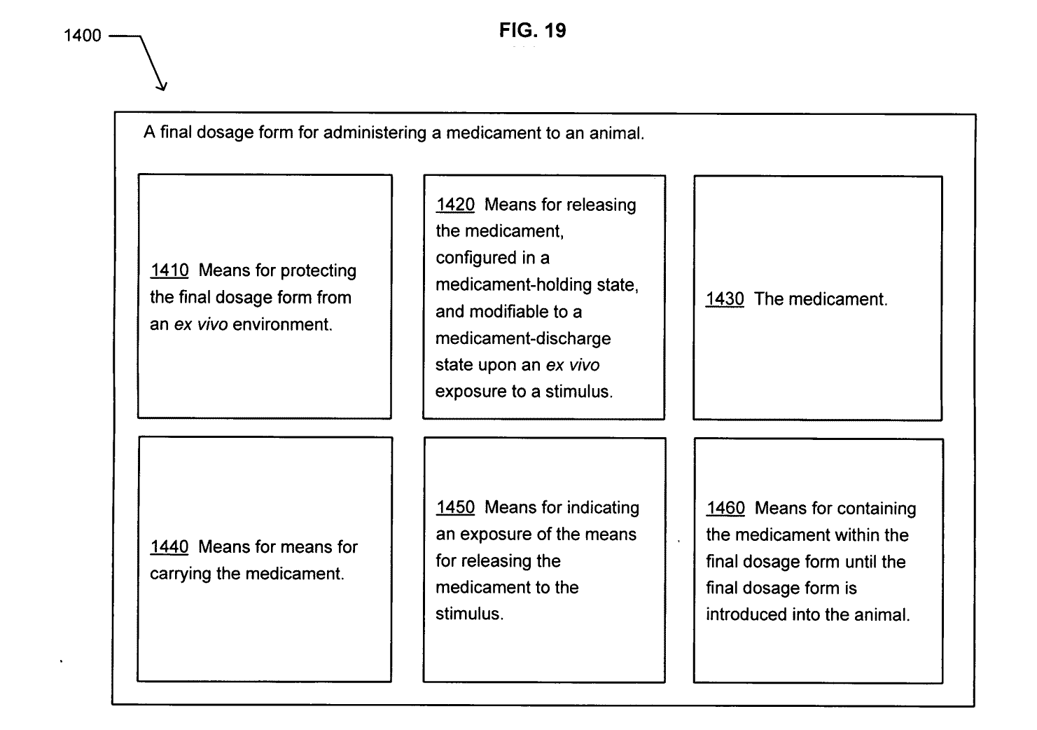

FIG. 19 illustrates an example final dosage form 1400 for administering a medicament to an animal. The final dosage includes means 1410 for protecting the final dosage form from an ex vivo environment. The final dosage form includes means 1420 for releasing the medicament, configured in a medicament-holding state, and modifiable ex vivo to a medicament-discharge state by an exposure to a stimulus, such as the stimulus 192. The final dosage form includes the medicament 1430.

In an embodiment, the final dosage form includes means 1440 for carrying the medicament. In an embodiment, the means 1440 for carrying the medicament is positioned substantially within the means 1410 for protecting the final dosage form. In an embodiment, the final dosage form includes means 1450 for indicating an exposure of the means for releasing the medicament to the stimulus. In an embodiment, the final dosage form includes means 1460 for containing the medicament within the final dosage form until the final dosage form is introduced into the animal.

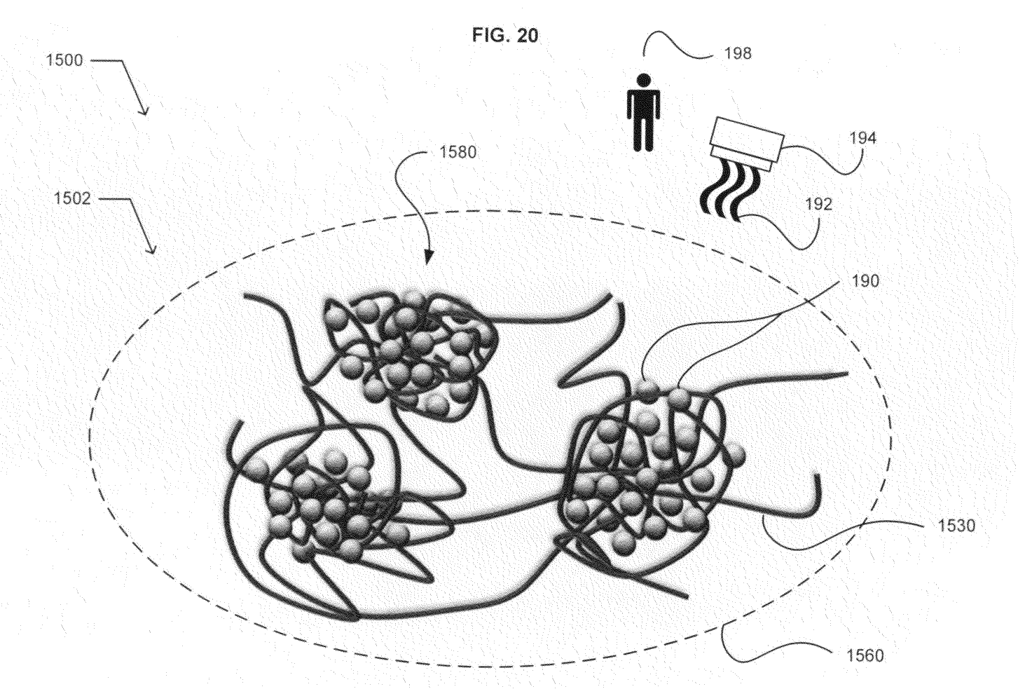

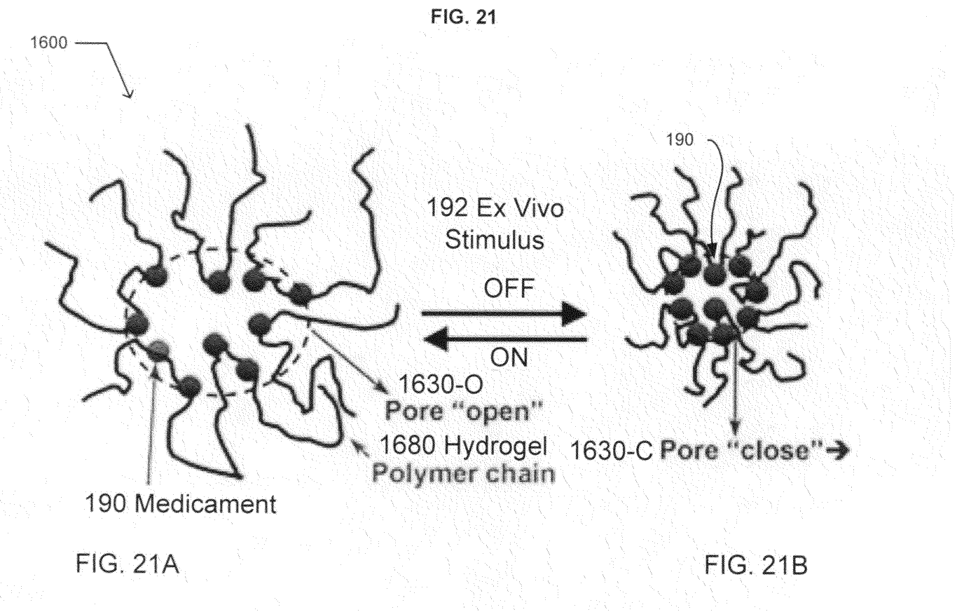

An embodiment provides a final dosage form for administering a medicament to an animal. In this embodiment, the final dosage form includes at least one particle or polymeric material respectively carrying at least one molecule of the medicament. The particle or polymeric material is configured in a medicament-retention state wherein the medicament is substantially not bioavailable to the animal if the final dosage form is administered to the animal. The particle or polymeric material is modifiable ex vivo by an exposure to the stimulus to a medicament-release state wherein the medicament is substantially bioavailable to the animal if the final dosage form is administered to the animal. As described subsequently, understanding of this embodiment may be facilitated by reference to FIG. 16, FIG. 20, and/or FIG. 21. In an embodiment, the particle or polymeric material may include at least one of the particle or polymeric materials previously described. In an embodiment, the particle or polymeric material includes an intelligent particle or polymeric material. In an embodiment, the particle or polymeric material includes a polymer matrix structure responsive to the exposure to a stimulus. In an embodiment, the particle or polymeric material includes at least one of a microparticle, a gel or a dendrimer based microparticle responsive to the exposure to a stimulus. In an embodiment, the particle or polymeric material includes at least one of a noisome, fibrin, polymeric micelle, microsome, cyclodextrin, polymer-medicament conjugate, or cellulose responsive to the exposure to a stimulus. In an embodiment, the particle or polymeric material includes at least one of a gel, a gel matrix, a natural gel, a synthetic gel, a colloid gel, or a hydrogel structure covalently bonded to the medicament using a photo labile bond and responsive to the exposure to a stimulus. A synthetic gel may include cellulose or polymers. In an embodiment, the particle or polymeric material includes at least one of a dendrimer, dendrimsome, dendromsome, dendron (partial dendrimer), or dendriplex material. In an embodiment, the particle or polymeric material includes at least one of an emulsion, nano-emulsion, or double emulsion. In an embodiment, the particle or polymeric material includes at least one of a lipid, cationic lipid, lipid micelle, liposome, lipospheres, acoustically active lipospheres, acoustically-active microbubbles conjugated to liposomes, lipid-coated microbubbles, cerasomes, magnetic liposomes, metallosomes, or a mimetic. Acoustically-active microbubbles conjugated to liposomes are described in A. Kheirolomoom, et al., Acoustically-active microbubbles conjugated to liposomes: characterization of a proposed drug delivery vehicle, J. CONTROL RELEASE 118 (3) (Apr. 23, 2007):275-84; Epub 2006 Dec. 23. A cerasomes may include liposomes with a silicate surface. A mimetic may include an artificial micelle or membrane.

FIG. 20 illustrates an example environment 1500 that includes a final dosage form 1502 configurable to administer a medicament to the animal 198. The final dosage form includes at least one molecule of the medicament 190. The final dosage form also includes at least one of a particle or polymeric material 1580, which is depicted as a gel. The particle or polymeric material has a characteristic response 1530 to the stimulus 192 that releases an engaged, retained, or entrapped at least one molecule of the medicament 190 from the particle or polymeric material. For example, a characteristic response of a particular particle or polymeric material may include a releasing bursting, expanding, cleaving, or degradation of the particular particle or polymeric material in response to a microwave stimulus.

The at least one particle or polymeric material 1580 respectively carries the at least one molecule of the medicament 190. The particle or polymeric material is configured in a medicament-retention state wherein the medicament is substantially not bioavailable to the animal 198 if the final dosage form is administered to the animal. The particle or polymeric material being modifiable ex vivo by an exposure to the stimulus 192 to carry the medicament in a medicament-release state wherein the medicament is substantially bioavailable to the animal if the final dosage form is administered to the animal.

FIG. 21 illustrates an example environment 1600 depicting retention and release states of particle or polymeric material 1680 (depicted as a hydrogel) responsive to an ex vivo stimulus 192. FIG. 21A illustrates a medicament-release state and FIG. 21B illustrates a medicament-retention state. Application of the stimulus ex vivo to the particle or polymeric material switches it from the medicament-retention state to the medicament-release state. For example, an ex vivo application of the stimulus to the hydrogel switches it from a pore “closed” state 1630-C to a pore “open” state 1630-O.

Returning to FIG. 16, FIG. 16 illustrates the particle or polymeric material 1180, which is depicted as a liposome. The particle or polymeric material carries the medicament 190. The particle or polymeric material is configured in a medicament-retention state wherein the medicament is substantially not bioavailable to the animal if the final dosage form is administered to the animal 198. The particle or polymeric material is modifiable ex vivo by an exposure to the stimulus 192 to carry the medicament in a medicament-release state wherein the medicament is substantially bioavailable to the animal if the final dosage form is administered to the animal.

Returning to FIG. 20, in an embodiment, the medicament 190 includes a pharmacologically-active agent. In an embodiment, the medicament includes at least one of an agent, treatment agent, drug, prodrug, therapeutic, nutraceutical, medication, vitamin, nutritional supplement, medicine, remedy, medicinal substance, or cosmetic.

In an embodiment, the particle or polymeric material 1580 carrying the medicament 190 includes a particle or polymeric material conjugated with the medicament. In an embodiment, the particle or polymeric material carrying the medicament includes a particle or polymeric material containing, intertwined, or bound with the medicament. In an embodiment, the particle or polymeric material carrying the medicament includes a particle or polymeric material entrapping the medicament.

In an embodiment, the particle or polymeric material 1180 carrying the medicament 190 includes a particle or polymeric material carrying the medicament 190 and configured in a medicament-withholding state. In the medicament-withholding state the medicament is substantially not bioavailable to the animal 198 if the final dosage form 1102 is administered to the animal 198. The particle or polymeric material is modifiable ex vivo by an exposure to the stimulus 192 to carry the medicament in a medicament-supplying state wherein the medicament is substantially bioavailable to the animal if the final dosage form is administered to the animal. In an embodiment, the particle or polymeric material includes a particle or polymeric material carrying the medicament and configured in a medicament-retention state. In the medicament-retention state the medicament is substantially not bioavailable to the animal if the final dosage form is administered to the animal. The particle or polymeric material is modifiable ex vivo upon at least one of a post-manufacture or a field exposure to the stimulus to a medicament-release state. In medicament-release state, the medicament is substantially bioavailable to the animal if the final dosage form is administered to the animal. In an embodiment, the particle or polymeric material includes a particle or polymeric material carrying the medicament and configured in a medicament-holding state. In the medicament-holding state the medicament is substantially not bioavailable to the animal if the final dosage form is administered to the animal. The particle or polymer material is modifiable ex vivo to an in vivo release-facilitation state by an exposure to the stimulus to a medicament-release state. In the in vivo release-facilitation state the medicament is substantially bioavailable to the animal if the final dosage form is administered to the animal.

In an embodiment, the particle or polymeric material 1580 includes a particle or polymeric material having a premodification characteristic that results in an insignificant uptake of the particle or polymeric material in the gastrointestinal tract of the animal 198. In an embodiment, the particle or polymeric material includes an intact particle or polymeric material having a premodification characteristic that results in an insignificant uptake of the particle or polymeric material in the gastrointestinal tract of the animal. In an embodiment, the particle or polymeric material includes at least one of a gel, gel matrix, or hydrogel structure covalently bonded to the medicament using a photo labile bond. An example of a medicament covalently bonded to a hydrogel using photo labile bonds, and the medicament is not be released unless the gel matrix is exposed to enough light to break the bonds is described in U.S. Pat. No. 6,985,770 to Nyhart, Jr. An example of a medicament conjugated to dendrimers using photocleavable or labile linkers, which can be made to release the drug using light or through acid cleavage is described in Y. Qiu, supra., and C. Henry, supra. In an embodiment, the particle or polymeric material includes at least one of a dendrimer, dendrimsome, or dendriplex material. Examples of photo-labile, radio-labile, and enzyme-labile dendrimers are described in U.S. Pat. No. 6,471,968 to Baker, et al.; and examples of photo labile biocompatible dendrimers made from poly(propyleneimine) (POPAM) interiors and poly(amidoamine) (PAMAM) are described in U.S. Pat. No. 7,078,461 to Tomalia, et al.

In an embodiment, the particle or polymeric material 1580 includes a liposome carrier entrapping the medicament and having an intact particle size resulting in an insignificant uptake in the gastrointestinal tract of the animal 198. In an embodiment, the particle or polymeric material includes a liposome carrier having a particle size of at least approximately three microns. An example of a liposome carrier having a particle size of at least approximately three microns resulting an insignificant uptake in the gastrointestinal tract of the animal is described in D. Deshmukh, Can intact liposomes be absorbed in the gut? LIFE SCI. 1981 Jan. 19; 28(3):239-42; See also, MARC J. OSTRO, LIPOSOMES: FROM BIOPHYSICS TO THERAPEUTICS 140 (1987); 42 J. PHARMACY AND PHARMACOLOGY 821-826 (1990); 86 INTER. J. PHARMACY 239-246 (1992); PHARMACEUTICAL PARTICULATE CARRIERS: THERAPEUTIC APPLICATIONS Ch. 4 (p. 65, and FIGS. 15 and 16 at page 92) (edited By Alain Rolland 1993). In an embodiment, the particle or polymeric material includes a liposome having a particle size of at least approximately four microns.

In an embodiment, the particle or polymeric material 1580 includes at least one of a nanoparticle, a microsphere, or a polymeric microsphere responsive to the exposure to the stimulus 192. In an embodiment, the particle or polymeric material includes a pharmaceutically-acceptable inert particle or polymeric material.

In an embodiment, the stimulus 192 includes a non-ionizing radiation stimulus. In an embodiment, the stimulus includes an electromagnetic radiation stimulus. In an embodiment, the stimulus includes at least one of a light radiation, terahertz radiation, microwave radiation, and radio wave radiation stimulus. In an embodiment, the stimulus includes a magnetic stimulus. In an embodiment, the stimulus includes an electric stimulus. In an embodiment, the stimulus includes an energetic stimulus. In an embodiment, the stimulus includes a chemical stimulus. In an embodiment, the stimulus includes at least one of a mechanical, heat, or pressure stimulus. In an embodiment, the stimulus includes at least one of an activation stimulus, or an actuation stimulus. In an embodiment, the stimulus includes at least one of at least one of a thermal, acoustic, or ultrasound stimulus. In an embodiment, the stimulus includes a stimulus facilitating a release of the medicament by at least one of an expansion of a gel, gel matrix, or hydrogel carrier. In an embodiment, the stimulus includes a stimulus facilitating a release of the medicament by at least one of an expansion of a gel, gel matrix, or hydrogel carrier to allow a diffusion and bioavailability of the medicament. In an embodiment, the stimulus includes a stimulus facilitating the release of the medicament from the particle or polymeric carrier by at least one of a bursting of a liposome material, formation of a pore in a liposome material, or an unpacking of the particle or polymeric material.

In an embodiment, the particle or polymeric material 1580 includes a particle or polymeric material carrying the medicament 190 and configured in a medicament-retention state wherein the medicament is substantially not bioavailable to the animal 198 if the final dosage form is administered to the animal. The particle or polymeric material modifiable ex vivo by an exposure to the stimulus 192 to carry the medicament in a medicament-release state allowing an in vivo release of the medicament if the final dosage form is administered to the animal.

In an embodiment, the particle or polymeric material 1580 includes a first particle or polymeric material carrying the medicament 190, and a second particle or polymeric material (not shown) carrying the first particle or polymeric material. The second particle or polymeric material is configured in a first particle or polymeric material-retention state wherein the first particle or polymeric material is substantially not bioavailable to the animal if the final dosage form is administered to the animal 198. The second particle or polymeric material is modifiable ex vivo by an exposure to the stimulus 192 to carry the medicament a first particle or polymeric material-release state wherein the first particle or polymeric material is substantially bioavailable to the animal if the final dosage form is administered to the animal.

In an embodiment, the final dosage form 1502 further includes a transport medium 1560 suitable for administering the particle or polymeric material 1580 carrying the medicament to the animal 198. For example, the transport medium may include a carrier, admixture, diluent, or excipient. In another example, the transport medium may include a polymer, such as a hydrogel. An example of a polymer transport medium is described in United States Patent Application Pub. 2008/0050445 by Alcantar. In an embodiment, the final dosage form further includes an indicator substance (not illustrated) configured to indicate an exposure of the particle or polymeric substance to the stimulus 192. In an embodiment, the final dosage form further includes an indicator substance (not illustrated) configured to visually indicate an exposure of the particle or polymeric substance to the stimulus 192.

An embodiment includes the final dosage form for administering a medicament 190 to the animal 198. In this embodiment, the final dosage form includes the medicament and a particle or polymeric material. The particle or polymeric material carries the medicament. The particle or polymeric material is configured in a medicament-retention state wherein the medicament is substantially not bioavailable to the animal if the final dosage form is administered to the animal. The particle or polymeric material is modifiable ex vivo by an exposure to a first stimulus to carry the medicament a first medicament-release state wherein the medicament has a first bioavailability to the animal if the final dosage form is administered to the animal. The particle or polymeric material is modifiable ex vivo by an exposure to a second stimulus to carry the medicament in a second medicament-release state wherein the medicament has a second bioavailability to the animal if the final dosage form is administered to the animal. Understanding of this embodiment may be facilitated by reference to the preceding description in conjunction with FIG. 16, FIG. 20, or FIG. 21. In an embodiment, the first bioavailability includes a first delivery rate of the medicament and the second bioavailability includes a second delivery rate of the medicament. For example, the particle or polymeric material may have a characteristic that include an adjustable pore size responsive to a temperature of the particle or polymeric material attained in response to a heat stimulus. The heat stimulus may include a microwave or a light source. The first stimulus may include an exposure of the particle or polymeric material to a first temperature, or to a given temperature for a first period of time. The second stimulus may include an exposure of the particle or polymeric material to a second temperature, or to a given temperature for a second period of time. For example, selective control of temperature-modulatable materials is described in G. Rao, et al., Synthesis of Smart Mesoporous Materials, MRS BULLETIN P7.8 (Spring 2003). For example, an adjustable porosity of an organic polymer membrane is described in R. Estrada, et al., Smart polymeric membranes with adjustable pore size, 52 International journal of polymeric materials 833-843 (No. 9, 2003). For example, a thermosensitive grafted polymeric system which can be triggered to release the loaded drug with an increase in temperature, induced by a magnetic thermal heating event, is described in I. Ankareddi, et al., Development of a Magnetically Triggered Drug Delivery System using Thermoresponsive Grafted Polymer Networks with Magnetic Nanoparticles, 2 NANOTECH 431-434 (Vol. 2, 2007). See also, R. Liburdy, et al., Microwave-triggered liposomal drug delivery: investigation of a model drug delivery system, Engineering in Medicine and Biology Society 1163-1164, (Vol. 4, November 1989) (Images of the Twenty-First Century, Proceedings of the Annual International Conference of the IEEE Engineering). In another example, the first stimulus may include a first ultrasound power density and the second stimulus may include a second ultrasound power density. For example, bioavailability of polymeric micelles as a variable function of ultrasound power density is at least suggested by A. Marin, et al., Acoustic activation of drug delivery from polymeric micelles: effect of pulsed ultrasound, 71 JOURNAL OF CONTROLLED RELEASE 239-249 (Issue 3, 28 Apr. 2001).

In an embodiment, the final dosage form 1502 configurable to administer a medicament to the animal 198 includes a containment element 1540. In an embodiment, the containment element 1540 may be substantially similar to the containment element 140 described in conjunction with FIG. 1. In an embodiment, the containment element 1540 includes a pH-sensitive component of a liposome. For example, S. Cho, et al, pH-dependent release property of dioleoylphosphatidyl ethanolamine liposomes, 25 KOREAN JOURNAL OF CHEMICAL ENGINEERING 390 (No. 2, 2008) describes a pH-sensitive liposome prepared by a detergent removal method that retains at neutral pH (6-8), and releases at pH 5. In an embodiment, the containment element includes tunable component of a liposome. For example, I. Hafez, et al., Tunable pH-Sensitive Liposomes Composed of Mixtures of Cationic and Anionic Lipids, 79 BIOPHYSICAL JOURNAL 1438 (Issue 3, 2000) describes a tunable liposome using cationic and anionic lipid mixtures (cholesteryl hemisuccinate (CHEMS) and N,N-dioleoyl-N,N-dimethylammonium chloride. In an embodiment, the containment element includes pH sensitive Chitosan and polyacrylamide copolymer hydrogels releasing contained substances upon pH changes. For example, as described in P. Bonina, et al., 19 JOURNAL OF BIOACTIVE AND COMPATIBLE POLYMERS 101 (No. 2, 2004). In an embodiment, the containment element includes a pH sensitive Chitosan and polyalkyleneoxide-maleic acid copolymer releasing substances on pH changes. For example, as described in T. Yoshizawa, et al., pH- and temperature-sensitive permeation through polyelectrolyte complex films composed of chitosan and polyalkyleneoxide-maleic acid copolymer, 241 JOURNAL OF MEMBRANE SCIENCE 347 (Issue 2, 2004). In an embodiment, the containment element includes an acrylic acid (AA) grafted onto porous polypropylene (PP) producing a pH sensitive membrane. For example, as described in Y. Wang, et al., pH sensitive polypropylene porous membrane prepared by grafting acrylic acid in supercritical carbon dioxide, 45 POLYMER 855 (No. 3, 2004).

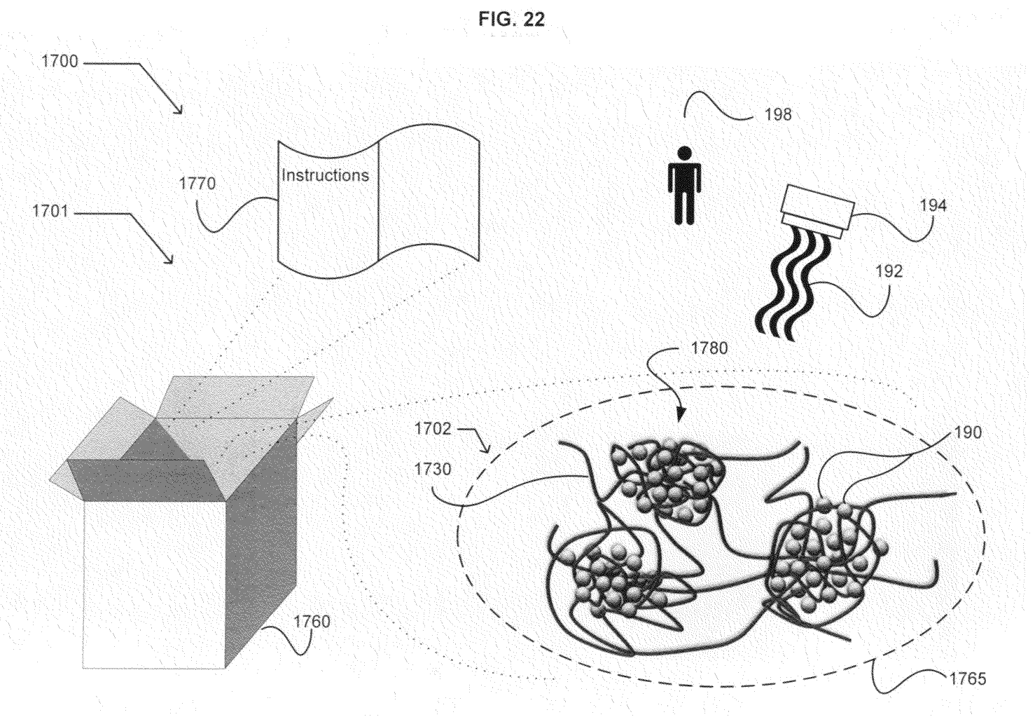

FIG. 22 illustrates an example environment 1700 that includes an article of manufacture 1701. The article of manufacture includes at least one final dosage form 1702 for administering the medicament 190 to the animal 198. The final dosage form includes the medicament, a particle or polymeric material 1780 carrying the medicament, and an instruction 1770. In an embodiment, the particle or polymeric material carrying the medicament may include the particle or polymeric material described in conjunction with FIG. 20. In an embodiment, the particle or polymeric material carrying the medicament may include a particle or polymeric material described in this paper. An embodiment of the particle or polymeric material carrying the medicament is depicted in FIG. 22 as a gel material for illustrative purposes. The particle or polymeric material has a characteristic response 1730 to the stimulus 192 that releases an engaged, retained, or entrapped at least one molecule of the medicament 190 from the particle or polymeric material. For example, a characteristic response of a particular particle or microparticle may include a releasing bursting, expanding, cleaving, or degradation of the particular particle or microparticle in response to a microwave stimulus.

The particle or polymeric material 1780 is in a medicament-retention state wherein the medicament is substantially not bioavailable to the animal 198 after administration of the final dosage form. The particle or polymeric material is modifiable ex vivo to a medicament-release state by an exposure to the stimulus 192 wherein the medicament is substantially bioavailable to the animal after administration of the final dosage form.

The instruction 1770 includes an instruction for the exposure of the particle or polymeric material 1780 to a human-initiated stimulus 192 sufficient to transform the particle or polymeric material to allow a discharge of at least a portion of the therapeutically effective amount of the medicament from the particle or polymeric carrier.

In an embodiment, the article of manufacture 1701 further includes a label associated with the at least one final dosage form 1702 or an insert into a package 1760 containing the at least one final dosage form, the insert providing the instructions 1770. In an embodiment, the final dosage form further includes a transport medium 1765 suitable for administering the particle or polymeric material 1780 carrying the medicament 190 to the animal 195. In an embodiment, the final dosage form further includes an indicator substance (not shown) configured to indicate an exposure of the particle or polymeric material to the stimulus 192.

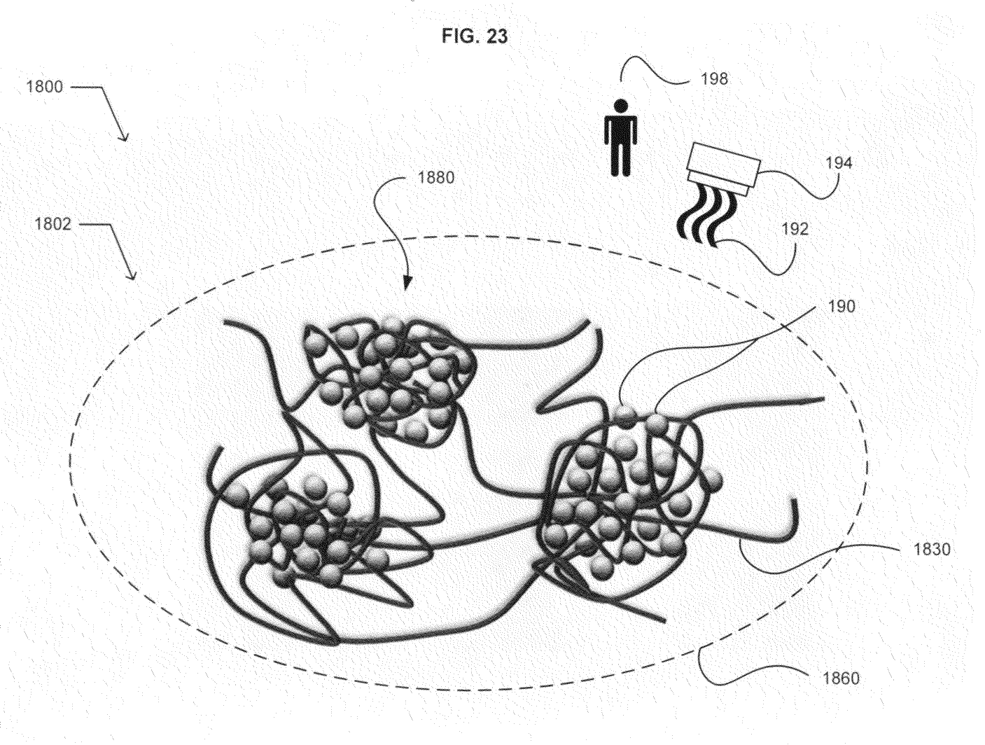

FIG. 23 illustrates an example environment 1800 that includes a final dosage form 1802 for administering the medicament 190 to the animal 198. The final dosage form includes at least one molecule of the medicament and a particle or polymeric carrier 1880 operable to bind the at least one molecule of the medicament, which is depicted as a gel. The particle or polymeric carrier is configured in a first medicament-bioavailability state, and modifiable ex vivo to a second medicament-bioavailability state by an exposure to a stimulus, illustrates as the stimulus 192. In an embodiment, the particle or polymeric carrier operable to bind the at least one molecule of the medicament has a characteristic response 1830 to a stimulus, such as the stimulus 192, that releases the bound at least one molecule of the medicament 190 from the particle or polymeric carrier. An example of such characteristic response is described in conjunction with FIG. 21. In another embodiment, the particle or polymeric carrier operable to bind the at least one molecule of the medicament has a characteristic response 1830 to a stimulus, such as the stimulus 192, that unbinds or releases the at least one molecule of the medicament 190 from the particle or polymeric carrier.

In an embodiment, the particle or polymeric carrier 1880 includes a pharmaceutically-acceptable inert particle or polymeric carrier operable to bind the at least one molecule of the medicament 190. In an embodiment, the particle or polymeric carrier includes a particle or polymeric carrier operable to engage, retain, or entrap at least one molecule of the medicament.

In an embodiment, the first medicament-bioavailability state is configured to retard medicament release in vivo and the second medicament-bioavailability state is configured to allow medicament release in vivo. In an embodiment, the first medicament-bioavailability state is configured to allow medicament release in vivo and the second medicament-bioavailability state is configured to retard medicament release in vivo.

In an embodiment, the particle or polymeric carrier 1860 includes a liposome carrier operable to bind the at least one molecule of the medicament 190 and having an intact particle size resulting in an insignificant uptake in the gastrointestinal tract of the animal 198. In an embodiment, liposome carrier operable to bind the at least one molecule of the medicament and having an intact particle size resulting in an insignificant uptake in the gastrointestinal tract of the animal includes a liposome carrier operable to bind the at least one molecule of the medicament and having an intact particle size of at least approximately one micron. For a description of an example, see, P. Hoet, et al., Nanoparticles—known and unknown health risks, 2 JOURNAL OF NANOBIOTECHNOLOGY 12, at section 4 (2004). In an embodiment, the liposome carrier operable to bind the at least one molecule of the medicament and having an intact particle size resulting in an insignificant uptake in the gastrointestinal tract of the animal includes a liposome carrier operable to bind the at least one molecule of the medicament and having an intact particle size of at least approximately three microns. In an embodiment, the liposome carrier operable to bind the at least one molecule of the medicament and having an intact particle size resulting in an insignificant uptake in the gastrointestinal tract of the animal includes a liposome carrier operable to bind the at least one molecule of the medicament and having an intact particle size of at least approximately four microns.

In an embodiment, the final dosage form 1802 further includes a transport medium 1860 suitable for administering to the animal 198 the particle or polymeric carrier 1880 holding the at least one molecule of the medicament 190. In an embodiment, the final dosage form of claim further includes an indicator substance (not shown) configured to visually indicate an exposure of the particle or polymeric carrier holding the at least one molecule of the medicament to the stimulus 192.

In an embodiment, the final dosage form 1802 configurable to administer a medicament to the animal 198 includes a containment element 1840. In an embodiment, the containment element 1580 may be substantially similar to the containment element 140 described in conjunction with FIG. 1. In an embodiment, the containment element 1540 described in conjunction with FIG. 20.

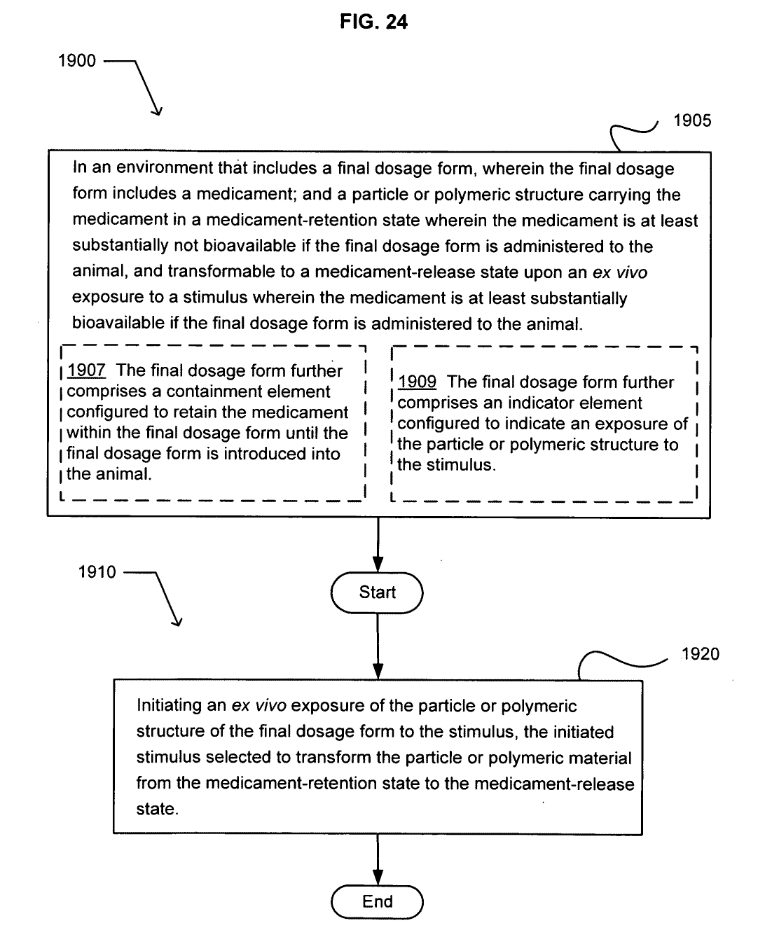

FIG. 24 illustrates an example environment 1900 that includes a final dosage form 1905 and an operational flow 1910. The final dosage form includes the medicament and a particle or polymeric material. The particle or polymeric material carries the medicament in the medicament-retention state wherein the medicament is substantially not bioavailable if the final dosage form is administered to the animal, such as the animal 198. The particle or polymeric material is transformable to the medicament-release state by the exposure to a stimulus, such as the stimulus 192, wherein the medicament is substantially bioavailable if the final dosage form is administered to the animal. In an embodiment, the final dosage form is at least similar to the final dosage form 1102 described in conjunction with FIG. 16. In an embodiment, the final dosage form is at least similar to the final dosage form 1502 described in conjunction with FIG. 20. In an embodiment, the final dosage form may is least similar to the final dosage form 1802 described in conjunction with FIG. 23. After a start operation, the operational flow includes an activation operation 1920. The activation operation includes initiating an exposure of the particle or polymeric material of the final dosage form to a stimulus, such as the stimulus 192 previously described. The initiated stimulus is selected to transform the particle or polymeric material from the medicament-retention state to the medicament-release state.

In an embodiment, the final dosage form 1905 further includes a containment element retaining the medicament within the final dosage form until the final dosage form is introduced into the animal. In an embodiment, the final dosage form further includes an indicator element configured to indicate an exposure of the particle or polymeric material to the stimulus.

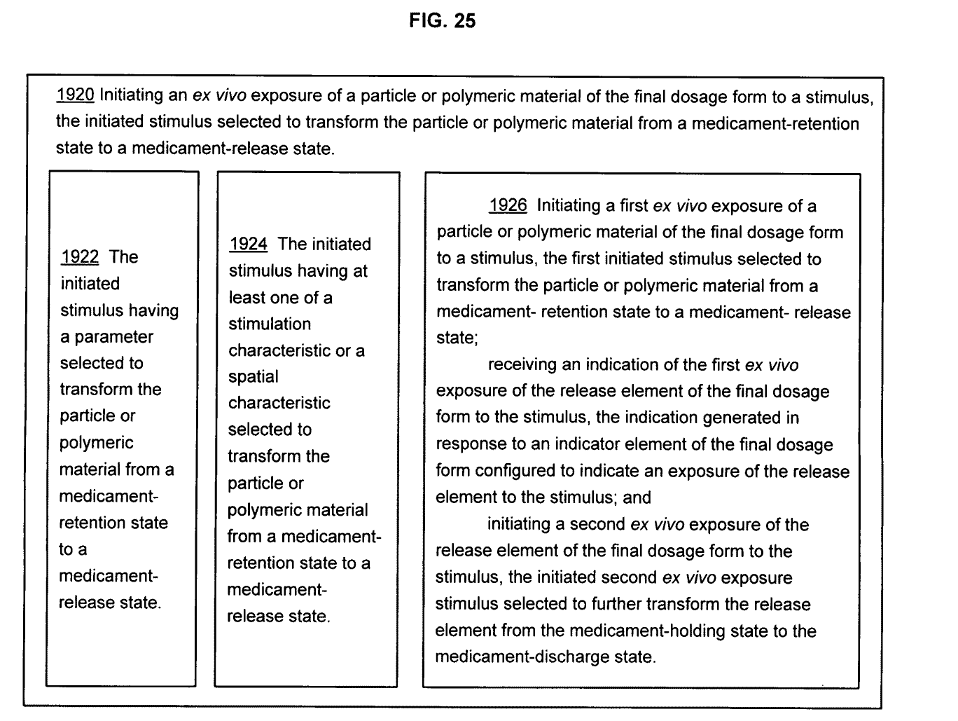

FIG. 25 illustrates alternative embodiments of the activation operation 1920 of FIG. 24. The activation operation may include an operation 1922, an operation 1924, or an operation 1926. The operation 1922 includes an initiated stimulus having a parameter selected to transform the particle or polymeric material from a medicament-retention state to a medicament-release state. The operation 1924 includes an initiated stimulus having at least one of a stimulation characteristic or a spatial characteristic selected to transform the particle or polymeric material from a medicament-retention state to a medicament-release state. The operation 1926 includes initiating a first exposure of a particle or polymeric material of the final dosage form to a stimulus, the first initiated stimulus selected to transform the particle or polymeric material from a medicament-retention state to a medicament-release state. The operation 1926 also includes receiving an indication of the first exposure of the release element of the final dosage form to the stimulus, the indication generated in response to an indicator element of the final dosage form configured to indicate an exposure of the release element to the stimulus. The operation 1926 further includes initiating a second exposure of the release element of the final dosage form to the stimulus, the initiated second exposure stimulus selected to further transform the release element from the medicament-holding state to the medicament-discharge state.

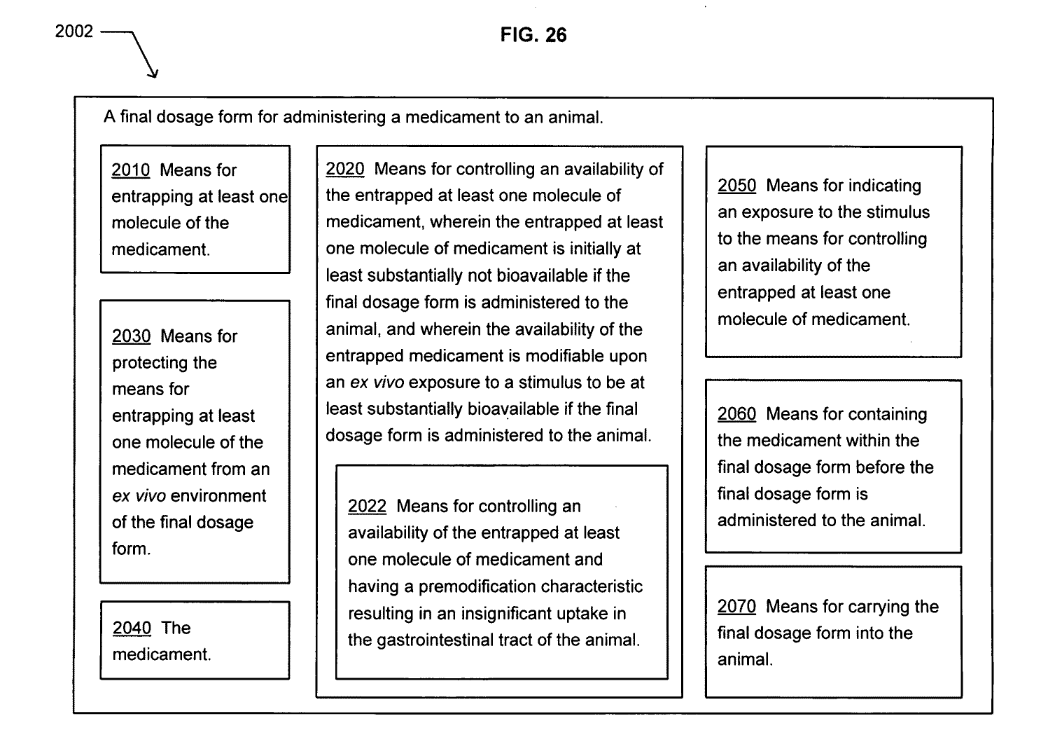

FIG. 26 illustrates an example embodiment of a final dosage form 2002 for administering a medicament, such as the medicament 190 as previously described, to an animal, such as the animal 198 as previously described. The final dosage form includes means 2010 for entrapping at least one molecule of the medicament. The final dosage form also includes means 2020 for controlling an availability of the entrapped at least one molecule of medicament, wherein the entrapped at least one molecule of medicament is initially substantially not bioavailable if the final dosage form is administered to the animal. The availability of the entrapped medicament is modifiable ex vivo by an exposure to a stimulus, such as the stimulus 192 described above, to be substantially bioavailable if the final dosage form is administered to the animal. The final dosage form further includes means 2030 for protecting the means 2010 for entrapping at least one molecule of the medicament from an ex vivo environment of the final dosage form. The final dosage form also includes the medicament 190.

In an embodiment, the means 2020 for controlling an availability of the entrapped at least one molecule of medicament includes means 2022 for controlling an availability of the entrapped at least one molecule of medicament and having a premodification characteristic resulting in an insignificant uptake in the gastrointestinal tract of the animal. In an embodiment, the final dosage form further includes means 2050 for indicating an exposure to the stimulus by the means for controlling an availability of the entrapped at least one molecule of medicament. In an embodiment, the final dosage form further includes means 2060 for containing the medicament within the final dosage form before the final dosage form is administered to the animal. In an embodiment, the final dosage form further includes means 2070 for carrying the final dosage form into the animal.

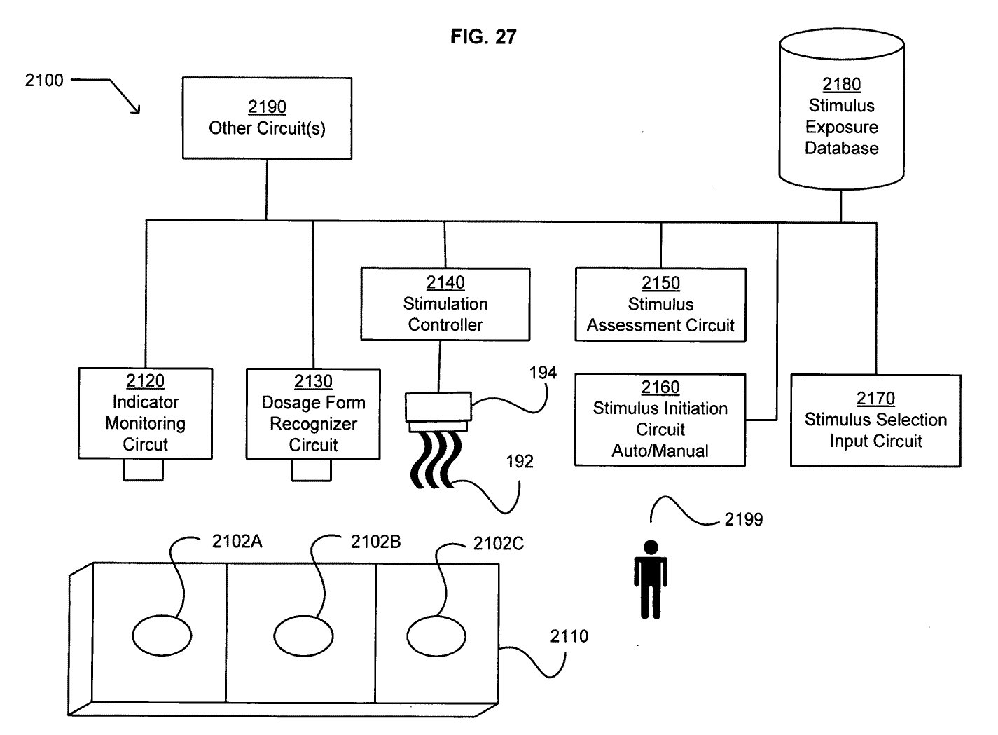

FIG. 27 illustrates an example system 2100 in which embodiments may be implemented. The system includes a final dosage form holder 2110 carrying at least one instance of a final dosage form, illustrated as final dosage forms 2102A-2102C. The system also includes the stimulus generator 194 having at least one controllable stimulus parameter, operable to generate the stimulus 192, and configured to direct the stimulus toward at least a portion of the final dosage form holder carrying at least one instance of a final dosage form. In an embodiment, the stimulus generator is operable to direct the stimulus toward a selectable at least a portion of the final dosage form holder carrying at least one instance of a final dosage form. The system further includes a stimulation controller 2140 operable to regulate the at least one controllable stimulus parameter. In an embodiment, the stimulation controller is operable to regulate the at least one controllable stimulus parameter by at least one of regulating the generator, by regulating a stimulus transmission pathway between the generator and the final dosage form holder, or by regulating which spatial portion of the dosage form receives the stimulus.

The system 2100 also includes a stimulation initiation circuit 2160 operable to initiate a stimulus having a selected stimulus parameter in response to a received input. In an embodiment, the stimulation initiation circuit is configured to transmit human perceivable indication of the assessed quality or quantity of the stimulus received by the at least one instance of the final dosage form. The received input by the stimulation initiation circuit may be received from an input by a human 2199, such as pharmacist or health care provider, or from a stimulus assessment circuit 2150. The stimulus assessment circuit is operable to monitor or quantify a quantity or quality of the stimulus received by the at least one instance of the final dosage form in response to data received from an indicator monitoring circuit 2120. The indicator monitoring circuit is configured to monitor an indicator substance portion of the at least one instance of the final dosage form. In an embodiment, the stimulus assessment circuit is configured to generate a signal usable to provide a human perceivable indication of the assessed quality or quantity of the stimulus received by the at least one instance of the final dosage form.

In an embodiment, the system 2100 includes a final dosage form recognizer circuit 2130 configured to generate data usable in distinguishing a particular type of final dosage form. In an embodiment, the system includes a stimulus selection input circuit 2170. In an embodiment, the stimulus selection input circuit is responsive to data generated by the final dosage form recognizer circuit. In an embodiment, the stimulus selection input circuit is responsive to a human 2199 initiated input.

In an embodiment, the final dosage form 2102 is substantially similar to the final dosage form 102 described in conjunction with FIG. 1. In an embodiment, the final dosage form 2102 is substantially similar to the final dosage form 202 described in conjunction with FIG. 2. In an embodiment, the final dosage form 2102 is substantially similar to the final dosage form 302 described in conjunction with FIG. 3. In an embodiment, the final dosage form 2102 is substantially similar to the final dosage form 402 described in conjunction with FIG. 4. In an embodiment, the final dosage form 2102 is substantially similar to the final dosage form 502 described in conjunction with FIG. 5. In an embodiment, the final dosage form 2102 is substantially similar to the final dosage form 602 described in conjunction with FIG. 6. In an embodiment, the final dosage form 2102 is substantially similar to the final dosage form 1002 described in conjunction with FIG. 15. In an embodiment, the final dosage form 2102 is substantially similar to the final dosage form 1102 described in conjunction with FIG. 16. In an embodiment, the final dosage form 2102 is substantially similar to the final dosage form 1202 described in conjunction with FIG. 17. In an embodiment, the final dosage form 2102 is substantially similar to the final dosage form 1502 described in conjunction with FIG. 20. In an embodiment, the final dosage form 2102 is substantially similar to the final dosage form 1702 described in conjunction with FIG. 22. In an embodiment, the final dosage form 2102 is substantially similar to the final dosage form 1802 described in conjunction with FIG. 23.

In an embodiment, the example operational flow 700 modulating a medicament-release characteristic of a final dosage form described in conjunction with FIG. 7 may be implemented using the system 2100. In an embodiment, the example operational flow 800 fulfilling a request specifying a dose of a medicament for an individual animal described in conjunction with FIG. 9 may be implemented using the system 2100. In an embodiment, the example operational flow 1300 modifying a medicament availability characteristic of a final dosage form described in conjunction with FIG. 18 may be implemented using the system 2100. In an embodiment, the example operation flow 1910 described in conjunction with FIG. 24 may be implemented using the system 2100.

All references cited herein are hereby incorporated by reference in their entirety or to the extent their subject matter is not inconsistent herewith.

In some embodiments, “configured” includes at least one of designed, set up, shaped, implemented, constructed, or adapted for at least one of a particular purpose, application, or function.

It will be understood that, in general, terms used herein, and especially in the appended claims, are generally intended as “open” terms (e.g., the term “including” should be interpreted as “including but not limited to,” the term “having” should be interpreted as “having at least,” the term “includes” should be interpreted as “includes but is not limited to,” etc.). It will be further understood that if a specific number of an introduced claim recitation is intended, such an intent will be explicitly recited in the claim, and in the absence of such recitation no such intent is present. For example, as an aid to understanding, the following appended claims may contain usage of introductory phrases such as “at least one” or “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a receiver” should typically be interpreted to mean “at least one receiver”); the same holds true for the use of definite articles used to introduce claim recitations. In addition, even if a specific number of an introduced claim recitation is explicitly recited, it will be recognized that such recitation should typically be interpreted to mean at least the recited number (e.g., the bare recitation of “at least two chambers,” or “a plurality of chambers,” without other modifiers, typically means at least two chambers).

Furthermore, in those instances where a phrase such as “at least one of A, B, and C,” “at least one of A, B, or C,” or “an [item] selected from the group consisting of A, B, and C,” is used, in general such a construction is intended to be disjunctive (e.g., any of these phrases would include but not be limited to systems that have A alone, B alone, C alone, A and B together, A and C together, B and C together, or A, B, and C together, and may further include more than one of A, B, or C, such as A₁, A₂, and C together, A, B₁, B₂, C₁, and C₂ together, or B₁ and B₂ together). It will be further understood that virtually any disjunctive word or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

The herein described aspects depict different components contained within, or connected with, different other components. It is to be understood that such depicted architectures are merely examples, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality. Any two components capable of being so associated can also be viewed as being “operably couplable” to each other to achieve the desired functionality. Specific examples of operably couplable include but are not limited to physically mateable or physically interacting components or wirelessly interactable or wirelessly interacting components.

With respect to the appended claims the recited operations therein may generally be performed in any order. Also, although various operational flows are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

While various aspects and embodiments have been disclosed herein, the various aspects and embodiments are for purposes of illustration and are not intended to be limiting, with the true scope and spirit being indicated by the following claims.

What is claimed is:

1. A final dosage form for administering a medicament to an animal, the final dosage form comprising:
the medicament; and
a particle or polymeric material carrying the medicament in a medicament-retention state wherein the medicament is substantially not bioavailable to the animal if the final dosage form is administered to the animal,
the particle or polymeric material being modifiable ex vivo by an exposure to a stimulus to carry the medicament in a medicament-release state wherein the medicament is substantially bioavailable to the animal if the final dosage form is administered to the animal.

2. The final dosage form of claim 1, wherein the medicament comprises a pharmacologically-active agent.

3. The final dosage form of claim 1, wherein the medicament comprises at least one of an agent, treatment agent, drug, prodrug, therapeutic, nutraceutical, medication, vitamin, nutritional supplement, medicine, remedy, medicinal substance, or cosmetic.

4. The final dosage form of claim 1, wherein the particle or polymeric material carrying the medicament comprises:
a particle or polymeric material conjugated with the medicament.

5. The final dosage form of claim 1, wherein the particle or polymeric material carrying the medicament comprises:
a particle or polymeric material containing, intertwined, or bound with the medicament.

6. The final dosage form of claim 1, wherein the particle or polymeric material carrying the medicament comprises:
a particle or polymeric material encapsulating the medicament.

7. The final dosage form of claim 1, wherein the stimulus includes at least one of light, radio, or electromagnetic wave stimulus.

8. The final dosage form of claim 1, wherein the stimulus includes at least one of a thermal, acoustic or ultrasound stimulus.

9. The final dosage form of claim 1, wherein the particle or polymeric material carrying the medicament in a medicament-retention state wherein the medicament is substantially not bioavailable to the animal if the final dosage form is administered to the animal, and modifiable ex vivo by an exposure to a stimulus to a medicament-release state wherein the medicament is substantially bioavailable to the animal if the final dosage form is administered to the animal comprises:
a particle or polymeric material carrying the medicament in a medicament-withholding state wherein the medicament is substantially not bioavailable to the animal if the final dosage form is administered to the animal, and modifiable ex vivo by an exposure to a stimulus to a medicament-supplying state wherein the medicament is substantially bioavailable to the animal if the final dosage form is administered to the animal.

10. The final dosage form of claim 1, wherein the particle or polymeric material carrying the medicament in a medicament-retention state wherein the medicament is substantially not bioavailable to the animal if the final dosage form is administered to the animal, and modifiable ex vivo by an exposure to a stimulus to a medicament-release state wherein the medicament is substantially bioavailable to the animal if the final dosage form is administered to the animal comprises:
a particle or polymeric material carrying the medicament in a medicament-retention state wherein the medicament is substantially not bioavailable to the animal if the final dosage form is administered to the animal, and modifiable ex vivo by at least one of a post-manufacture or a field exposure to a stimulus to a medicament-release state wherein the medicament is substantially bioavailable to the animal if the final dosage form is administered to the animal.

11. The final dosage form of claim 1, wherein the particle or polymeric material carrying the medicament in a medicament-retention state wherein the medicament is substantially not bioavailable to the animal if the final dosage form is administered to the animal, and modifiable ex vivo by an exposure to a stimulus to a medicament-release state wherein the medicament is substantially bioavailable to the animal if the final dosage form is administered to the animal comprises:
a particle or polymeric material carrying the medicament in a medicament-holding state wherein the medicament is substantially not bioavailable to the animal if the final dosage form is administered to the animal, and modifiable to a in vivo release-facilitation state by an ex vivo exposure to a stimulus to a medicament-release state wherein the medicament is substantially bioavailable to the animal if the final dosage form is administered to the animal.

12. The final dosage form of claim 1, wherein the particle or polymeric material comprises:
a particle or polymeric material having a premodification characteristic that results in an insignificant uptake of the particle or polymeric material in the gastrointestinal tract of the animal.

13. The final dosage form of claim 1, wherein the polymeric material comprises an intelligent polymer.

14. The final dosage form of claim 1, wherein the particle or polymeric material comprises a polymer matrix structure.

15. The final dosage form of claim 1, wherein the particle or polymeric material comprises at least one of a microparticle, a gel or a dendrimer based microparticle.

16. The final dosage form of claim 1, wherein the particle or polymeric material comprises:
at least one of a noisome, fibrin, polymeric micelle, microsome, cyclodextrin, polymer-medicament conjugate, or cellulose responsive to the ex vivo exposure to a stimulus.

17. The final dosage form of claim 1, wherein the particle or polymeric material includes at least one of a gel, a gel matrix, a natural gel, a synthetic gel, a colloid gel, or a hydrogel structure covalently bonded to the medicament using a photo labile bond and responsive to the ex vivo exposure to a stimulus.

18. The final dosage form of claim 1, wherein the particle or polymeric material includes at least one of a dendrimer, dendrimsome, dendromsome, dendron, or dendriplex material.

19. The final dosage form of claim 1, wherein the particle or polymeric material includes at least one of an emulsion, nano-emulsion, or double emulsion.

20. The final dosage form of claim 1, wherein the particle or polymeric material includes at least one of a lipid, cationic lipid, lipid micelle, liposome, lipospheres, acoustically active lipospheres, acoustically-active microbubbles conjugated to liposomes, lipid-coated microbubbles, cerasomes, magnetic liposomes, metallosomes, or a mimetic.

21. The final dosage form of claim 1, wherein the particle or polymeric material comprises:
a liposome carrier entrapping the medicament and having an intact particle size resulting in an insignificant uptake in the gastrointestinal tract of the animal.

22. The final dosage form of claim 1, wherein the particle or polymeric material includes a liposome carrier having a particle size of at least approximately three microns.

23. The final dosage form of claim 1, wherein the particle or polymeric material includes a liposome having a particle size of at least approximately four microns.

24. The final dosage form of claim 1, wherein the particle or polymeric material includes a microparticle material.

25. The final dosage form of claim 1, wherein the particle material comprises:
at least one of a nanoparticle, a microsphere, or a polymeric microsphere responsive to the ex vivo exposure to a stimulus.

26. The final dosage form of claim 1, wherein the particle or polymeric material includes a pharmaceutically-acceptable inert particle or polymeric material.

27. The final dosage form of claim 1, wherein the particle or polymeric material carrying the medicament in a medicament-retention state wherein the medicament is substantially not bioavailable to the animal if the final dosage form is administered to the animal, and modifiable ex vivo by an exposure to a stimulus to a medicament-release state wherein the medicament is substantially bioavailable to the animal if the final dosage form is administered to the animal comprises:
a particle or polymeric material carrying the medicament in a medicament-retention state wherein the medicament is substantially not bioavailable to the animal if the final dosage form is administered to the animal, and modifiable ex vivo by an exposure to a non-ionizing radiation stimulus to a medicament-release state wherein the medicament is substantially bioavailable to the animal if the final dosage form is administered to the animal

28. The final dosage form of claim 1, wherein the particle or polymeric material carrying the medicament in a medicament-retention state wherein the medicament is substantially not bioavailable to the animal if the final dosage form is administered to the animal, and modifiable ex vivo by an exposure to a stimulus to a medicament-release state wherein the medicament is substantially bioavailable to the animal if the final dosage form is administered to the animal comprises:
a particle or polymeric material carrying the medicament in a medicament-retention state wherein the medicament is substantially not bioavailable to the animal if the final dosage form is administered to the animal, and modifiable ex vivo by an exposure to an electromagnetic radiation stimulus to a medicament-release state wherein the medicament is substantially bioavailable to the animal if the final dosage form is administered to the animal

29. The final dosage form of claim 1, wherein the particle or polymeric material carrying the medicament in a medicament-retention state wherein the medicament is substantially not bioavailable to the animal if the final dosage form is administered to the animal, and modifiable ex vivo by an exposure to a stimulus to a medicament-release state wherein the medicament is substantially bioavailable to the animal if the final dosage form is administered to the animal comprises:
a particle or polymeric material carrying the medicament in a medicament-retention state wherein the medicament is substantially not bioavailable to the animal if the final dosage form is administered to the animal, and modifiable ex vivo by an exposure to at least one of a light radiation, terahertz radiation, microwave radiation, and radio wave radiation stimulus to a medicament-release state wherein the medicament is substantially bioavailable to the animal if the final dosage form is administered to the animal.

30. The final dosage form of claim 1, wherein the particle or polymeric material carrying the medicament in a medicament-retention state wherein the medicament is substantially not bioavailable to the animal if the final dosage form is administered to the animal, and modifiable ex vivo by an exposure to a stimulus to a medicament-release state wherein the medicament is substantially bioavailable to the animal if the final dosage form is administered to the animal comprises:
a particle or polymeric material carrying the medicament in a medicament-retention state wherein the medicament is substantially not bioavailable to the animal if the final dosage form is administered to the animal, and modifiable ex vivo by an exposure to a magnetic stimulus to a medicament-release state wherein the medicament is substantially bioavailable to the animal if the final dosage form is administered to the animal.

31. The final dosage form of claim 1, wherein the particle or polymeric material carrying the medicament in a medicament-retention state wherein the medicament is substantially not bioavailable to the animal if the final dosage form is administered to the animal, and modifiable ex vivo by an exposure to a stimulus to a medicament-release state wherein the medicament is substantially bioavailable to the animal if the final dosage form is administered to the animal comprises:
a particle or polymeric material carrying the medicament in a medicament-retention state wherein the medicament is substantially not bioavailable to the animal if the final dosage form is administered to the animal, and modifiable ex vivo by an exposure to an electric stimulus to a medicament-release state wherein the medicament is substantially bioavailable to the animal if the final dosage form is administered to the animal.

32. The final dosage form of claim 1, wherein the particle or polymeric material carrying the medicament in a medicament-retention state wherein the medicament is substantially not bioavailable to the animal after administration of the final dosage form and modifiable ex vivo to a medicament-release state by an exposure to a stimulus wherein the medicament is substantially bioavailable to the animal after administration of the final dosage form comprises:
a particle or polymeric material carrying the medicament in a medicament-retention state wherein the medicament is substantially not bioavailable to the animal after administration of the final dosage form and modifiable ex vivo to a medicament-release state wherein the medicament is substantially bioavailable to the animal after administration of the final dosage form by an exposure to an energetic stimulus.

33. The final dosage form of claim 1, wherein the particle or polymeric material carrying the medicament in a medicament-retention state wherein the medicament is substantially not bioavailable to the animal after administration of the final dosage form and modifiable ex vivo to a medicament-release state by an exposure to a stimulus wherein the medicament is substantially bioavailable to the animal after administration of the final dosage form comprises:
a particle or polymeric material carrying the medicament in a medicament-retention state wherein the medicament is substantially not bioavailable to the animal after administration of the final dosage form and modifiable ex vivo to a medicament-release state wherein the medicament is substantially bioavailable to the animal after administration of the final dosage form by an exposure to a chemical stimulus.

34. The final dosage form of claim 1, wherein the particle or polymeric material carrying the medicament in a medicament-retention state wherein the medicament is substantially not bioavailable to the animal if the final dosage form is administered to the animal, and modifiable ex vivo by an exposure to a stimulus to a medicament-release state wherein the medicament is substantially bioavailable to the animal if the final dosage form is administered to the animal comprises:
a particle or polymeric material carrying the medicament in a medicament-retention state wherein the medicament is substantially not bioavailable to the animal if the final dosage form is administered to the animal, and modifiable ex vivo by an exposure to at least one of a mechanical, heat, or pressure stimulus to a medicament-release state wherein the medicament is substantially bioavailable to the animal if the final dosage form is administered to the animal.

35. The final dosage form of claim 1, wherein the particle or polymeric material carrying the medicament in a medicament-retention state wherein the medicament is substantially not bioavailable to the animal if the final dosage form is administered to the animal, and modifiable ex vivo by an exposure to a stimulus to a medicament-release state wherein the medicament is substantially bioavailable to the animal if the final dosage form is administered to the animal comprises:
a particle or polymeric material carrying the medicament in a medicament-retention state wherein the medicament is substantially not bioavailable to the animal if the final dosage form is administered to the animal, and modifiable ex vivo by an exposure to at least one of an activation stimulus, or an actuation stimulus to a medicament-release state wherein the medicament is substantially bioavailable to the animal if the final dosage form is administered to the animal.

36. The final dosage form of claim 1, wherein the particle or polymeric material carrying the medicament in a medicament-retention state wherein the medicament is substantially not bioavailable to the animal if the final dosage form is administered to the animal, and modifiable ex vivo by an exposure to a stimulus to a medicament-release state wherein the medicament is substantially bioavailable to the animal if the final dosage form is administered to the animal comprises:
a particle or polymeric material carrying the medicament in a medicament-retention state wherein the medicament is substantially not bioavailable to the animal if the final dosage form is administered to the animal, and modifiable ex vivo by an exposure to at least one of at least one of a thermal, acoustic, or ultrasound stimulus to a medicament-release state wherein the medicament is substantially bioavailable to the animal if the final dosage form is administered to the animal.

37. The final dosage form of claim 1, wherein the particle or polymeric material carrying the medicament in a medicament-retention state wherein the medicament is substantially not bioavailable to the animal if the final dosage form is administered to the animal, and modifiable ex vivo by an exposure to a stimulus to a medicament-release state wherein the medicament is substantially bioavailable to the animal if the final dosage form is administered to the animal comprises:
a particle or polymeric material carrying the medicament in a medicament-retention state wherein the medicament is substantially not bioavailable to the animal if the final dosage form is administered to the animal, and modifiable ex vivo by an exposure to a stimulus to a medicament-release state wherein the medicament is substantially bioavailable to the animal if the final dosage form is administered to the animal, the stimulus facilitating a release of the medicament by at least one of an expansion of a gel, gel matrix, or hydrogel carrier.

38. The final dosage form of claim 1, wherein the particle or polymeric material carrying the medicament in a medicament-retention state wherein the medicament is substantially not bioavailable to the animal if the final dosage form is administered to the animal, and modifiable ex vivo by an exposure to a stimulus to a medicament-release state wherein the medicament is substantially bioavailable to the animal if the final dosage form is administered to the animal comprises:
a particle or polymeric material carrying the medicament in a medicament-retention state wherein the medicament is substantially not bioavailable to the animal if the final dosage form is administered to the animal, and modifiable ex vivo by an exposure to a stimulus to a medicament-release state wherein the medicament is substantially bioavailable to the animal if the final dosage form is administered to the animal, the stimulus facilitating the release of the medicament from the particle or polymeric carrier by at least one of a bursting of a liposome material, formation of a pore in a liposome material, or an unpacking of the particle or polymeric material.

39. The final dosage form of claim 1, wherein the particle or polymeric material carrying the medicament in a medicament-retention state wherein the medicament is substantially not bioavailable to the animal if the final dosage form is administered to the animal, and modifiable ex vivo by an exposure to a stimulus to a medicament-release state wherein the medicament is substantially bioavailable to the animal if the final dosage form is administered to the animal comprises:
a particle or polymeric material carrying the medicament in a medicament-retention state wherein the medicament is substantially not bioavailable to the animal if the final dosage form is administered to the animal, and modifiable ex vivo by an exposure to a stimulus to a medicament-release state allowing an in vivo release of the medicament if the final dosage form is administered to the animal.

40. The final dosage form of claim 1, wherein the particle or polymeric material carrying the medicament in a medicament-retention state wherein the medicament is substantially not bioavailable to the animal if the final dosage form is administered to the animal, and modifiable ex vivo by an exposure to a stimulus to a medicament-release state wherein the medicament is substantially bioavailable to the animal if the final dosage form is administered to the animal comprises:
a first particle or polymeric material carrying the medicament; and
a second particle or polymeric material carrying the first particle or polymeric material, configured in a first particle or polymeric material-retention state wherein the first particle or polymeric material is substantially not bioavailable to the animal if the final dosage form is administered to the animal, and modifiable ex vivo by an exposure to a stimulus to a first particle or polymeric material-release state wherein the first particle or polymeric material is substantially bioavailable to the animal if the final dosage form is administered to the animal.

41. The final dosage form of claim 1, further comprising:
a transport medium suitable for delivering the particle or polymeric material carrying the medicament to the animal.

42. The final dosage form of claim 1, further comprising:
an indicator substance configured to indicate an exposure of the particle or polymeric substance to the stimulus.

43. A final dosage form for administering a medicament to an animal, the final dosage form comprising:
the medicament; and
a particle or polymeric material carrying the medicament in a medicament-retention state wherein the medicament is substantially not bioavailable to the animal if the final dosage form is administered to the animal,
the particle or polymeric material being modifiable ex vivo by an exposure to a first stimulus to carry the medicament in a first medicament-release state wherein the medicament has a first bioavailability to the animal if the final dosage form is administered to the animal, and
the particle or polymeric material being modifiable ex vivo by an exposure to a second stimulus to a second medicament-release state, wherein the medicament has a second bioavailability to the animal if the final dosage form is administered to the animal.
